(19) United States
(12) Patent Application Publication
(10) Pub. No.: US 2018/0016333 A1
(43) Pub. Date: Jan. 18, 2018

(54) PHARMACEUTICAL FORMULATIONS FOR ANTI-TNF-ALPHA ANTIBODIES

(71) Applicants: MABXIENCE, S.A., Montevidei (UY); MABXIENCE RESEARCH, S.L., Madrid (ES)

(72) Inventors: Carlos BAÑADO, Madrid (ES); Cédric BÈS, Madrid (ES); Orla MCGARVEY, Madrid (ES)

(21) Appl. No.: 15/546,598

(22) PCT Filed: Jan. 28, 2016
(86) PCT No.: PCT/EP2016/051850

§ 371 Date: Jul. 26, 2017

(30) Foreign Application Priority Data
Jan. 28, 2015 (EP) 15152923.7

(51) Int. Cl. C07K 16/24; A61K 47/12; A61K 9/00; A61K 47/26; A61K 39/395

(52) U.S. Cl. CPC C07K 16/241; A61K 47/26; A61K 39/39591; A61K 9/0019; A61K 47/12; C07K 2317/21; C07K 2317/76

(57) ABSTRACT

The present invention relates to aqueous stable antibody compositions suitable for long term storage, in particular comprising antibodies against Tumor Necrosis Factor alpha (anti-TNFα). More specifically, it provides an aqueous composition comprising: —an anti-TNFα antibody; —a buffer selected from the list consisting of an acetate buffer, a citrate buffer, and a citrate-acetate buffer; and —an excipient, wherein said excipient is at least selected from a disaccharide, a sugar alcohol and a combination thereof; wherein when the buffer comprises or consists of an acetate buffer, said composition comprises a disaccharide at a concentration of less than 240 mM; wherein when the buffer comprises or consists of a citrate or a citrate-acetate buffer, said composition comprises a sugar alcohol at a concentration from 50 mM to 300 mM; and wherein the pH of the composition is from pH 4.0 to pH 7.0. It further relates to the pharmaceutical use of said composition, in particular in the treatment of inflammatory and immune system mediated diseases associated to an increase of TNFα. In addition, the invention relates to methods for obtaining said composition and devices comprising thereof.

### FIELD OF INVENTION

The present invention relates to aqueous stable antibody compositions suitable for long term storage, in particular comprising antibodies against Tumor Necrosis Factor alpha (anti-TNFα). It further relates to the pharmaceutical use of said compositions, in particular in the treatment of inflammatory and immune system mediated diseases. In addition, the invention relates to methods for obtaining said composition and devices comprising thereof.

### BACKGROUND OF THE INVENTION

Therapeutic polypeptide preparations are often stored prior to use. Polypeptides, however, are unstable if stored in aqueous form for an extended period of time. An alternative to relying on aqueous storage is to prepare a dry lyophilized form of a polypeptide, although, reconstitution of a dried polypeptide often results in aggregation or denaturation. This aggregation of polypeptides is undesirable as it may result in immunogenicity.

Tumor necrosis factor alpha (TNFα) is a naturally occurring cytokine that is involved in normal inflammatory and immune responses. Elevated levels of TNFα are found in the synovial fluid of patients with rheumatoid arthritis, juvenile idiopathic arthritis, psoriatic arthritis and ankylosing spondylitis and play an important role in both the pathologic inflammation and the joint destruction that are hallmarks of these diseases, increased levels of TNFα being also found in psoriasis plaques.

A commercially available recombinant human antibody that binds to soluble human TNFα is known as HUMIRA® (adalimumab). Adalimumab interferes with TNFα by acting as a TNF inhibitor and blocking its interaction with the p55 and p75 cell surface TNF receptors. Adalimumab also modulates biological responses that are induced or regulated by TNF, including changes in the levels of adhesion molecules responsible for leukocyte migration (ELAM-1, VCAM-1, and ICAM-1 with an IC50 of 0.1-0.2 nM). This human IgG1 anti-TNFα antibody is currently formulated as an aqueous composition for subcutaneous injection with a sodium phosphate and sodium citrate buffer system and mannitol as stabilizing agent (see EP1528933-B1).

Anti-TNFα antibody aqueous compositions similar to that disclosed in EP1528933-B1 are known in the art, namely comprising an organic acid/salt buffer system; and a disaccharide or a sugar alcohol.

For instance, EP2471554-A1, discloses the screening of 42 different formulations. Notably, 7 organic acids/salts (glutamate, aconitic acid, ascorbic acid, malic acid, tartaric acid, adipic acid, and citric acid) and 5 stabilisers (mannitol, glycine, trehalose, sorbitol and sucrose) were tested, all formulated with 105 mM NaCl, 0.1% polysorbate 80 and pH around 5.2.

WO2004/016286 discloses (see, Table 1.1) an anti-TNFα antibody aqueous pharmaceutical composition comprising mannitol, polysorbate 80, sodium chloride and a citrate-phosphate buffer system.

Furthermore, EP2471554-A1, WO2013/186230 and WO2014/039903 disclose formulations wherein the buffer system is an acetate buffer, with mannitol used for stability purposes.

On the other hand, WO2013/186230 and WO 2013/164837, disclose anti-TNFα antibody formulations wherein the buffer system is an acetate buffer and trehalose is used as stabilizing agent. WO2013/186230 discloses specifically a trehalose concentration of 240 mM (see F8 in example 1), whereas WO 2013/164837 a trehalose concentration of 140 mM (see, example 10). In both instances, said formulation further comprises arginine which is also used as stabilizing agent.

The present invention addresses the problem of providing a novel stable anti-TNFα antibody liquid formulation suitable for pharmaceutical administration which enables long term storage. The inventors have obtained such stable aqueous compositions as disclosed herein.

### SUMMARY OF THE INVENTION

The first aspect of the present invention relates to aqueous composition comprising:
an anti-TNFα antibody;
a buffer selected from the list consisting of an acetate buffer, a citrate buffer, and a citrate-acetate buffer; and
an excipient, wherein said excipient is at least selected from a disaccharide, a sugar alcohol and a combination thereof;
wherein when the buffer comprises or consists of an acetate buffer, said composition comprises a disaccharide at a concentration of less than 240 mM;
wherein when the buffer comprises or consists of a citrate or a citrate-acetate buffer, said composition comprises a sugar alcohol at a concentration from 50 mM to 300 mM; and
wherein the pH of the composition is from pH 4.0 to pH 7.0.

A second aspect of the present invention relates to the use of said aqueous composition under the first aspect of the invention as a pharmaceutical composition. It further relates to a pharmaceutical composition of the invention and to devices comprising thereof that can be used to deliver it.

A third aspect of the invention relates to said composition or pharmaceutical composition for use in a method of treating inflammatory and/or immune system mediated diseases, more specifically for use in a method of treating inflammatory and/or immune system mediated diseases associated with an increase of TNFα. A related aspect, is directed to methods of treating inflammatory and/or immune system mediated diseases, more specifically to methods of treating inflammatory and/or immune system mediated diseases associated with an increase of TNFα, with a composition or pharmaceutical composition of the present invention.

In an additional aspect, the invention relates to a method of manufacturing an aqueous composition according to the preceding aspects which comprises the steps of:
preparing a buffer selected from the list consisting of an acetate buffer, a citrate buffer, or a citrate-acetate buffer at the required pH,
adding a disaccharide and/or sugar alcohol, and optionally, a surfactant and/or salt,
adding an aqueous solution, preferably water, to the final volume and when necessary adjusting the pH,
incorporating an anti-TNFα antibody to the composition.

### BRIEF DESCRIPTION OF THE DRAWINGS

FIG. 1 shows the protein concentration measurements (Absorbance at 280 nm) at all times (0 and 14 days) and conditions (4° C., 25° C., 40° C., 3 times freezing/thawing and 3 days in agitation).

FIG. 2 shows hydrodynamic size measured with Dynamic Light Scattering at all times (0 and 14 days) and conditions (4° C., 25° C., 40° C., 3 times freezing/thawing and 3 days in agitation).

FIG. 3 shows SDS-PAGE gels stained with Coomassie in non reduced conditions incubated at all temperatures: −20° C., 25° C., 50° C., 3 times freezing/thawing and 3 days in agitation at times 0 and 14 days. In (A), F1 and F4 samples, in (B) F1 and F2 samples, in (C) F2 and F3 samples, in (D) F3 and F4 samples, in (E) F8 and F9 samples and in (F) F8 and F9 samples.

FIG. 4 shows SDS-PAGE gels stained with Coomassie in reduced conditions incubated at all temperatures: −20° C., 25° C., 50° C., 3 times freezing/thawing and 3 days in agitation at times 0 and 14 days. In (A), F1 and F4 samples, in (B) F1 and F2 samples, in (C) F2 and F3 samples, in (D) F3 and F4 samples, in (E) F8 and F9 samples and in (F) F8 and F9 samples.

### DETAILED DESCRIPTION OF THE INVENTION

The first aspect of the present invention relates to aqueous composition comprising:
an anti-TNFα antibody;
a buffer selected from the list consisting of an acetate buffer, a citrate buffer, and a citrate-acetate buffer; and
an excipient, wherein said excipient is at least selected from a disaccharide, a sugar alcohol and a combination thereof;
wherein when the buffer comprises or consists of an acetate buffer, said composition comprises a disaccharide at a concentration of less than 240 mM;
wherein when the buffer comprises or consists of a citrate or a citrate-acetate buffer, said composition comprises a sugar alcohol at a concentration from 50 mM to 300 mM; and wherein the pH of the composition is from pH 4.0 to pH 7.0.

As used herein, the term “composition” or “compositions” may refer to a formulation(s) comprising an antibody prepared such that it is suitable for injection and/or administration into an individual in need thereof. A “composition” may also be referred to as a “pharmaceutical composition.” In certain embodiments, the compositions provided herein are substantially sterile and do not contain any agents that are unduly toxic or infectious to the recipient. Further, as used herein, a solution or “aqueous composition” may mean a fluid (liquid) preparation that contains water, optionally in combination with other mutually miscible solvents (e.g. water-soluble organic solvents), and one or more chemical substances dissolved therein.

Further, as used herein, words of approximation such as, without limitation, “about”, “around”, “approximately” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by ±1, 2, 3, 4, 5, 6, 7, 10, 12 or 15%. Accordingly, the term “about” may mean the indicated value±5% of its value, preferably the indicated value±2% of its value, most preferably the term “about” means exactly the indicated value (±0%).

As used herein, the term “antibody” or “antibodies” may refer to an immunoglobulin or an antigen-binding fragment thereof. Unless otherwise specified, the term includes, but is not limited to, polyclonal, monoclonal, monospecific, polyspecific, humanized, human, single-chain, chimeric, synthetic, recombinant, hybrid, mutated, grafted, and in vitro generated antibodies. The antibody can include a constant region, or a portion thereof, such as those encoded by the kappa, lambda, alpha, gamma, delta, epsilon and mu constant region genes. For example, heavy chain constant regions of the various isotypes can be used, including: IgG1, IgG2, IgG3, IgG4, IgM, IgA1, IgA2, IgD, and IgE. By way of example, the light chain constant region can be kappa or lambda.

The term “antigen-binding fragment” as used herein may refer to a part of an antibody molecule that comprises amino acids responsible for the specific binding between antibody and antigen. For certain antigens, the antigen-binding fragment may only bind to a part of the antigen. The part of the antigen that is specifically recognized and bound by the antibody is referred to as the “epitope” or “antigenic determinant.” Antigen-binding fragments include Fab (Fragment antigen-binding); a F(ab′)2 fragment, a bivalent fragment having two Fab fragments linked by a disulfide bridge at the hinge region; Fv fragment; a single chain Fv fragment (scFv) see e.g., Bird et al. (1988) Science 242:423-426; and Huston et al. (1988) Proc. Natl. Acad. Sci. USA 85:5879-5883); a Fd fragment having the two VH and CH1 domains; dAb (Ward et al., (1989) Nature 341:544-546), and other antibody fragments that retain antigen-binding function. The Fab fragment has VH-CH1 and VL-CL domains covalently linked by a disulfide bond between the constant regions. The Fv fragment is smaller and has VH and VL domains non-covalently linked. To overcome the tendency of non-covalently linked domains to dissociate, a scFv can be constructed. The scFv contains a flexible polypeptide that links (1) the C-terminus of VH to the N-terminus of VL, or (2) the C-terminus of VL to the N-terminus of VH. These antibody fragments are obtained using conventional techniques known to those with skill in the art, and the fragments are evaluated for function in the same manner as are intact antibodies.

Preferably, said anti-TNFα antibody is an antibody against human TNFα. The term “human TNFα” (abbreviated herein as hTNFα, or simply hTNF), as used herein, is intended to refer to a human cytokine that exists as a 17 kD secreted form and a 26 kD membrane associated form, the biologically active form of which is composed of a trimer of noncovalently bound 17 kD molecules. The structure of hTNFα is described further in, for example, Pennica, D., et al. (1984) Nature 312:724-729; Davis, J. M., et al. (1987) Biochemistry 26: 1322-1326; and Jones, E. Y., et al. (1989) Nature 338:225-228. The term human TNFα is intended to include recombinant human TNFα (rhTNFα), which can be prepared by standard recombinant expression methods or purchased commercially (R&D Systems, Catalog No. 210-TA, Minneapolis, Minn.).

In a particular embodiment, said anti-TNFα antibody is a human antibody, preferably a human anti-hTNFα antibody. The term “human antibody” as used herein, is intended to include antibodies having variable and constant regions derived from human germline immunoglobulin sequences. The human antibodies may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

Preferably, said human antibody is a recombinant antibody. The expression “recombinant human antibody” as used herein, may refer to all human antibodies that are prepared, expressed, created or isolated by recombinant means, such as antibodies expressed using a recombinant expression vector transfected into a host cell, antibodies isolated from a recombinant combinatorial human antibody library, antibodies isolated from an animal (e.g., a mouse) that is transgenic for human immunoglobulin genes (see e.g., Taylor, L. D., et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain aspects, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In a preferred embodiment, the anti-TNFα antibody is adalimumab. Adalimumab, commercialized under the trade name Humira®, is a recombinant human anti-hTNFα IgG1 antibody. This antibody is also known as D2E7. It consists of 1330 amino acids and has a molecular weight of approximately 148 kilodaltons. (http://www.drugbank.ca/drugs/DB00051). Adalimumab has been described and claimed in U.S. Pat. No. 6,090,382. The term “adalimumab” is also intended to include the so-called “biosimilar” and “biobetter” versions of the active adalimumab protein present in commercially available Humira®.

As used herein, “biosimilar” (of an approved reference product/biological drug, such as a protein therapeutic, an antibody, etc.) refers to a biologic product that is similar to the reference product based upon data derived from (a) analytical studies that demonstrate that the biological product is highly similar to the reference product notwithstanding minor differences in clinically inactive components; (b) animal studies (including the assessment of toxicity); and/or (c) a clinical study or studies (including the assessment of immunogenicity and pharmacokinetics or pharmacodynamics) that are sufficient to demonstrate safety, purity, and potency in one or more appropriate conditions of use for which the reference product is approved and intended to be used and for which approval is sought for the biological product. In one embodiment, the biosimilar biological product and reference product utilize the same mechanism or mechanisms of action for the condition or conditions of use prescribed, recommended, or suggested in the proposed labelling, but only to the extent the mechanism or mechanisms of action are known for the reference product. In one embodiment, the condition or conditions of use prescribed, recommended, or suggested in the labelling proposed for the biological product have been previously approved for the reference product. In one embodiment, the route of administration, the dosage form, and/or the dose of the biological product are the same as those of the reference product. In one embodiment, the facility in which the biological product is manufactured, processed, packed, or held meets standards designed to assure that the biological product continues to be safe, pure, and potent. The reference product may be approved in at least one of the U.S., Europe, or Japan.

A recombinant anti-TNFα antibody (e.g. adalimumab) can be produced by standard methods known in the art for the production of antibodies. For example, U.S. Pat. Nos. 6,090,382 and 8,216,583 describe various methods that a skilled artisan could use to prepare adalimumab for use in the formulations of the present invention. These methods are incorporated by reference herein. For example, adalimumab can be prepared by recombinant expression of immunoglobulin light and heavy chain genes in a host cell.

Systems for cloning and expression of a polypeptide in a variety of different host cells are well known in the art, these may be from eukaryotic or prokaryotic organisms (i.e., bacterial cells, such as E. coli). For cells suitable for producing antibodies, see for instance Gene Expression Systems, Academic Press, eds. Fernandez et al, 1999. Preferably, said host cells are eukaryotic cells. The most common eukaryotic expression platforms currently include yeast (e.g., Pichia pastoris and Saccharomyces cerevisiae), baculovirus expression vector systems (Autographa californica multiple nuclear polyhedrosis virus and insect cell hosts Spodoptera frugiperda or Trichoplusia ni), and mammalian cell systems (including a variety of transformed and/or genetically modified cell lines). More preferably, said cells are mammalian cells, including simian, human, dog and rodent cells. Examples of human cells are PER.C6 cells (WO01/38362), MRC-5 (ATCC CCL-171), WI-38 (ATCC CCL-75), HEK-293 cells (ATCC CRL-1573), HeLa cells (ATCC CCL2), and fetal rhesus lung cells (ATCC CL-160). Examples of non-human primate cells are Vero cells (ATCC CCL81), COS-1 cells (ATCC CRL-1650) or COS-7 cells (ATCC CRL-1651). Examples of dog cells are MDCK cells (ATCC CCL-34). Examples of rodent cells are hamster cells, such as BHK21-F, HKCC cells, or CHO cells. Any protein compatible expression system may be used to produce the anti-TNFα antibody used in the composition of the invention (e.g. adalimumab). Suitable expression systems include transgenic animals described in Gene Expression Systems, Academic Press, eds. Fernandez et al., 1999.

In a preferred embodiment, said recombinant anti-TNFα antibody (e.g. adalimumab) is produced by recombinant DNA technology in a mammalian host cell, preferably in a Chinese hamster ovary (CHO) mammalian cell expression system which is the host cell used for the production of Humira®.

Purification of the expressed anti-TNFα antibody can be performed by any standard method. Methods for polypeptide isolation and/or purification are well known in the art (see for instance, Isolation and Purification of Proteins, Feb. 5, 2003 by CRC Press, ISBN 9780824707262). Procedures for purification of polypeptides initially depend on the site of expression of the protein. Some proteins are secreted into the cell culture media; others are intracellular proteins. In the second instance, the first step of a purification process involves lysis of the cell, which can be done by a variety of methods, including mechanical shear, osmotic shock, or enzymatic treatments and generally, cell debris is removed by differential centrifugation or by filtration. Typically, when the protein is secreted into the medium, supernatants are first concentrated using standard polypeptide concentration filters. Protease inhibitors can also be added to inhibit proteolysis and antibiotics can be included to prevent the growth of microorganisms.

The anti-TNFα antibody can be purified using, for example, hydroxyapatite chromatography, gel electrophoresis, dialysis, and affinity chromatography, and any combination of known or yet to be discovered purification techniques, including but not limited to Protein A chromatography, fractionation on an ion-exchange column, ethanol precipitation, reverse phase HPLC, chromatography on silica, chromatography on heparin SEPHAROSET®, an anion or cation exchange resin chromatography (such as a polyaspartic acid column), chromatofocusing, SDS-PAGE, and ammonium sulfate precipitation.

The concentration of the anti-TNFα antibody or human anti-hTNFα antibody or adalimumab is not particularly limited. For instance, it may be from 5 mg/ml to 500 mg/mL, from 7.5 mg/ml to 250 mg/mL, from 10 mg/ml to 200 mg/mL, from 10 mg/ml to 150 mg/mL or from 10 mg/ml to 100 mg/mL. In a particular embodiment, said antibody concentration is from 40 to 200 mg/mL, such as 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195 or 200 mg/mL. In another particular embodiment, the anti-TNFα antibody or human anti-hTNFα antibody or adalimumab concentration is from 10 to 100 mg/mL, preferably between 20 and 60 mg/mL, more preferably from 25 to 55 mg/mL and even more preferably the concentration is of about 40 mg/mL, about 45 mg/mL or about 50 mg/mL. Most preferably, the concentration is of about 10 mg/mL, about 50 mg/mL, about 100 mg/mL, about 150 mg/ml or about 200 mg/ml.

The composition according to the first aspect of the invention comprises an aqueous buffer which is selected from the group consisting of an acetate buffer, a citrate buffer, a phosphate buffer and a combination thereof, such as from the group consisting of an acetate buffer, a citrate buffer, and a phosphate buffer. The term “acetate buffer”, “citrate buffer” and “phosphate buffer” as used herein might refer to a buffer system comprising and organic acid (namely acetic acid, citric acid and phosphoric acid, respectively) and/or a salt thereof (i.e., comprising the corresponding conjugate base, namely an acetate ion, citrate ion and phosphate ion, respectively).

In a particular embodiment, said aqueous buffer is a phosphate buffer, such as sodium phosphate or potassium phosphate. In another embodiment, the buffer is selected from the list consisting of an acetate buffer, a citrate buffer, and a citrate-acetate buffer. In a preferred embodiment, the buffer is selected from the list consisting of an acetate buffer, a citrate buffer, and a citrate-acetate buffer with the proviso that the composition of the invention does not comprise a citrate-phosphate buffer. In another preferred embodiment, the buffer is selected from the list consisting of an acetate buffer, a citrate buffer, and a citrate-acetate buffer with the proviso that the composition does not comprise a phosphate buffer. In a further embodiment, the buffer does not consist of a sole histidine buffer or comprises histidine in combination with buffers other than citrate or acetate buffer.

In another embodiment, said aqueous buffer is a citrate buffer. Illustrative non-limiting examples of a citrate buffer are sodium citrate or potassium citrate. In a further embodiment said aqueous buffer is an acetate buffer. Illustrative non-limiting examples of an acetate buffer are sodium acetate, sodium diacetate, magnesium acetate, potassium acetate, ammonium acetate, tris-(hydroxymethyl)-aminomethane (tris) acetate, or histidine acetate. In a preferred embodiment, said acetate buffer is histidine acetate. In another preferred embodiment, said aqueous buffer is sodium acetate. In a further preferred embodiment, said aqueous buffer is a citrate-acetate buffer. Regardless of the buffer used in the composition, alone or in combination, the concentration thereof is preferably from 5 mM to 100 mM, more preferably from 10 mM to 50 mM, most preferably in the range of 15 mM to 30 mM. In a more preferred embodiment said concentration is about 10 mM or about 20 mM or about 25 mM. Particularly preferred buffers are acetate buffers, more preferably histidine acetate or sodium acetate, in a concentration from 10 mM to 50 mM, preferably of about 20 mM. Other particularly preferred buffers are citrate or citrate-acetate buffers in a concentration from 10 mM to 50 mM, preferably of about 20 mM.

The pH of the composition might be from pH 4.0 to pH 7.0, preferably from pH 4.4 to pH 6.5, more preferably from pH 5.0 to pH 6.0, being possible any pH selected from 5.1, 5.2, 5.3, 5.4, 5.5, 5.6, 5.7, 5.8 and 5.9. In one embodiment, the pH of the composition is from pH 4.0 to pH 5.7, being possible any pH selected from 4.1, 4.2, 4.3, 4.4, 4.5, 4.6, 4.7, 4.8, 4.9, 5.0, 5.1, 5.2, 5.3, 5.4, 5.5, 5.6, preferably the pH of the composition is about pH 5.2 and the buffer is a citrate buffer (preferably, sodium citrate) or an acetate buffer (preferably, sodium acetate or histidine acetate). In another embodiment, the buffer is a citrate buffer (preferably, sodium citrate) and the pH is from pH 4.4 to pH 5.2, being possible any pH selected from 4.5, 4.6, 4.7, 4.8, 4.9, 5.0, and 5.1, preferably the pH of the composition is about pH 4.4 (preferably pH 4.42) or about pH 5.2. In a further embodiment, the pH of the composition is from pH 5.5 to pH 6.5, being possible any pH selected from 5.6, 5.7, 5.8, 5.9, 6.0, 6.1, 6.2, 6.3 and 6.4, preferably the pH of the composition is about pH 6.0 and the buffer is a phosphate buffer (preferably, sodium phosphate).

The composition according to the first aspect of the invention comprises a pharmaceutically acceptable excipient, wherein said excipient is at least selected from the list consisting of a disaccharide, a sugar alcohol and a combination thereof. In a preferred embodiment, said disaccharide, sugar alcohol or combination thereof, is used as a stabilizing agent. Preferably, the concentration of said disaccharide, sugar alcohol or combination thereof is of less than 300 mM, preferably of less than 240 mM, more preferably of less than 140 mM. In a preferred embodiment, when the buffer comprises or consists of an acetate buffer, said composition comprises a disaccharide (preferably trehalose, sucrose or a combination thereof) at a concentration of less than 140 mM.

Examples of suitable disaccharides for the compositions of the present invention include lactose, trehalose, sucrose, and combinations thereof. Additional examples of disaccharides that can be used in some embodiments of this invention include at least one of maltose, isomaltose, cellobiose, isosaccharose, isotrehalose, sorbose, turanose, melibiose, gentiobiose, and mixtures thereof.

Preferably, the disaccharide is trehalose, sucrose or a combination thereof. For example, the aqueous composition comprises trehalose and/or sucrose at a concentration from 5 to 290 mM, such as from 30 mM to 230 mM, such as from 40 mM to 220 mM, or such as from 45 mM to 200 mM. In a specific embodiment, the trehalose and/or sucrose concentration is less than 240 mM, preferably less than 140 mM, more preferably less than 135 mM, for example within the range from 40 mM to 130 mM. In another specific embodiment, the sucrose and/or trehalose concentration is higher than 50 mM, such as higher than 55 mM, for example within the range of 55 mM to 135 mM, preferably from 80 to 120 mM, such as from 65 mM to 100 mM.

In a preferred embodiment, the disaccharide is trehalose at a concentration from 40 mM to 130 mM, preferably from 50 mM to 75 mM and more preferably at a concentration of about 55 mM, about 60 mM, about 65 mM, or about 70 mM of trehalose, preferably in the form of trehalose dihydrate. In another preferred embodiment, the excipient is sucrose at a concentration from 40 mM to 130 mM, preferably sucrose is present in the range of 80 mM to 120 mM, more preferably from 90 mM to 110 mM. In a more preferred embodiment the concentration of sucrose is of about 95 mM, about 100 mM, or about 105 mM. In a specific embodiment, the aqueous composition comprises a combination between sucrose and trehalose, wherein the concentrations of trehalose and sucrose is less than 240 mM, preferably less than 140 mM, more preferably less than 135 mM, for example within the range from 40 mM to 130 mM. In another specific embodiment, the sucrose and trehalose concentration is higher than 50 mM, such as higher than 55 mM, for example within the range of 55 mM to 135 mM, preferably from 60 to 120 mM, such as from 65 mM to 100 mM. In a preferred embodiment, the excipient is a combination between sucrose and trehalose, where the trehalose concentration is from 55 mM to 75 mM (preferably about 65 mM) and the sucrose concentration is from 80 mM to 120 mM (preferably about 100 mM).

In another particular embodiment, said excipient is a sugar alcohol. Examples of sugar alcohols suitable for the present invention include mannitol, xylitol, erythritol, threitol, ribitol, myoinisitol, galactitol, sorbitol, glycerol and the like. Preferably, said sugar alcohol is found at a concentration from 50 mM to 300 mM, such as from 150 mM to 300 mM, such as from 60 mM to 275 mM, such as from 65 mM to 150 mM, or such as from 65 mM to 85 mM. In a preferred embodiment, said sugar alcohol is present at a concentration above 240 mM, more preferably from 250 to 300, for example of about 275 mM. In another preferred embodiment, said sugar alcohol is present at a concentration below 240 mM, preferably at a concentration below 200 mM. In a more preferred embodiment, said sugar alcohol is present at a concentration from 40 mM to 195 mM, preferably from 50 mM to 100 mM, more preferably from 55 mM to 90 mM, such as at a concentration of about 65 mM, of about 75 mM or of about 85 mM. In a preferred embodiment, said excipient is mannitol. The term “mannitol” as used herein may refer to D-mannitol, an hexahydric alcohol related to mannose and is isomeric with sorbitol. In a more preferred embodiment, said excipient is mannitol in a concentration of about 275 mM, about 85 mM, about 75 mM, about 65 mM or about 55 mM.

In a preferred embodiment, when the buffer is an acetate buffer, said aqueous composition comprises a disaccharide, preferably at a concentration of less than 240 mM, more preferably less than 140 mM, most preferably less than 135 mM, for example at a concentration from 40 mM to 130 mM, such as from 50 mM to 75 mM, or such as from 80 mM to 120 mM. In another preferred embodiment, when the buffer is a citrate or phosphate buffer, said aqueous composition comprises a sugar alcohol (preferably mannitol), preferably at a concentration above 240 mM, more preferably from 250 to 300, for example about 275 mM. In another preferred embodiment, when the buffer is a citrate or phosphate buffer, said aqueous composition comprises mannitol at a concentration below 240 mM, preferably at a concentration below 200 mM. In a more preferred embodiment, mannitol is present at a concentration from 40 mM to 195 mM, preferably from 50 mM to 100 mM, more preferably from 55 mM to 90 mM, such as at a concentration of about 65 mM, of about 75 mM or of about 85 mM.

The aqueous composition of the present invention is a stable composition. As used herein, a “stable composition” may refer to a formulation in which the antibody therein essentially retains its physical stability and/or chemical stability and/or biological activity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10:29-90 (1993), for example. Stability can be measured at a selected temperature for a selected time period. Preferably, the formulation is stable at room temperature (about 25° C.) or at 40° C. for at least 14 days, for at least 1 month or for at least 3 months, and/or stable at about 2-8° C. (preferably at 4° C.) for at least 1 month, for at least 3 months, for at least 6 months, for at least 1 year or for at least 2 years. Furthermore, the formulation is preferably stable following freezing (to, e.g., −80° C.) and thawing of the formulation hereinafter referred to as a “freeze/thaw cycle”, preferably after 3 freeze/thaw cycles and/or after agitation, preferably after 3 days in agitation at room temperature.

An antibody “retains its physical stability” in a pharmaceutical formulation if it shows substantially no signs of aggregation, precipitation and/or denaturation upon visual examination of color and/or clarity, or as measured by UV light scattering (e.g. Dynamic Light Scattering) or by size exclusion chromatography.

An antibody “retains its chemical stability” in a pharmaceutical formulation, if the chemical stability at a given time is such that the antibody is considered to still retain its biological activity as defined below. Chemical stability can be assessed by detecting and quantifying chemically altered forms of the antibody. Chemical alteration may involve size modification (e.g. clipping) which can be evaluated using size exclusion chromatography, SDS-PAGE and/or matrix assisted laser desorption ionization/time-of flight mass spectrometry (MALDI/TOF MS), for example. Other types of chemical alteration include charge alteration (e.g. occurring as a result of deamidation) which can be evaluated, for example, by ion exchange chromatography.

An antibody “retains its biological activity” in a pharmaceutical formulation, if the antibody in a pharmaceutical formulation is biologically active for its intended purpose. For example, biological activity is retained if the reduction of the biological activity of the antibody in the pharmaceutical formulation is within about 30%, about 20%, about 10%, about 5% or less of the biological activity present at the time the pharmaceutical formulation was prepared.

The composition of the present invention preferably comprises as a further excipient a surfactant. The inclusion of surfactants may protect proteins at potentially destabilizing interfaces, against surfaces encountered during processing, and against the alteration of their thermodynamic conformational stability. Surfactants are well known in the art, such as polysorbate or polyoxyl 40 stearate. Other possible surface-active agents include phospholipids, such as a lecithin; polyoxyethylene-polyoxypropylene copolymers, such as a Pluronic surfactant; polyoxyethylene esters of 12-hydroxysteraric acid, such as a Solutol surfactant; ethoxylates of cholesterol, such as diacyl glycerol, dialkyl glycerol; bile salts, such as sodium cholate, sodium deoxycholate; sucrose esters, such as sucrose monolaurate, sucrose monooleate; polyvinyl pyrrolidone (PVP); or polyvinyl alcohol (PVA). In a particular embodiment, the surfactant is polysorbate, such as for example polysorbate 20, polysorbate 40, polysorbate 60 or polysorbate 80. Preferably, the polysorbate is polysorbate 80 (polyoxyethylene (20) sorbitan monooleate). The surfactant is preferably present at a concentration from 0.01% w/v to 1% w/v, more preferably from 0.05% w/v to 0.2% w/v, most preferably of about 0.1% w/v. In a preferred embodiment the polysorbate is polysorbate 80 and is present at a concentration of 0.1% w/v (0.76 mM).

The composition of the present invention preferably comprises as a further excipient a salt. The salt concentration may be of more than 100 mM or from 80 to 130 mM. Preferably the salt concentration is from 90 to 120 mM, such as from 90 to 115 mM or from 100 to 115 mM, specifically about 105 mM or about 110 mM. In a preferred embodiment, the salt concentration (preferably NaCl) is about 105 mM.

Regardless of the concentration, the salt is preferably sodium chloride, although other salts such as potassium chloride, sodium citrate, magnesium sulphate, calcium chloride, sodium hypochlorite, sodium nitrate, mercury sulphide, sodium chromate and magnesium dioxide can also be used. Preferably, said aqueous composition is isotonic. The term “isotonic” as used herein may mean that the osmolality is close to the physiological osmolality in the human body, thus leading to more suitable compositions to be used in parenteral administration, e.g. subcutaneous administration. Isotonic formulations will generally have an osmotic pressure from about 250 to 350 mOsm/L, preferably about 290 mOsm/L. Isotonicity can be measured using a vapor pressure or ice-freezing type osmometer, for example.

In a preferred embodiment, the composition of the present invention consists of the above-described ingredients, i.e. it is a composition consisting of adalimumab, an aqueous citrate, acetate, or citrate-acetate buffer, a sugar alcohol or disaccharide, and optionally a salt, and/or a surfactant. In a further preferred embodiment, the composition of the present invention consists of the above-described ingredients, i.e. it is a composition consisting of adalimumab, an aqueous citrate, acetate, or citrate-acetate buffer, a sugar alcohol or disaccharide, salt, and a surfactant. The preferred embodiments of the chemical nature and the concentrations of these ingredients are described above.

In another embodiment, the composition according to the present invention may comprise further excipients, either replacing the salt and the surfactant, or in addition to the salt and the surfactant. One or more other pharmaceutically acceptable carriers, excipients or stabilizers such as those described in Remington's Pharmaceutical Sciences 17th edition. (1985) may be included in the formulation provided that they do not adversely affect the desired characteristics of the formulation. Acceptable carriers, excipients or stabilizers are nontoxic to recipients at the dosages and concentrations employed and include additional buffering agents; co-solvents; antioxidants including ascorbic acid and methionine; chelating agents such as EDTA; metal complexes (e.g. Zn-protein complexes); salt-forming counterions such as sodium, amino acid polymers; sulfur-containing reducing agents, such as urea, glutathione, thioctic acid, sodium thioglycolate, thioglycerol, α-monothioglycerol and sodium thiosulfate; low molecular weight polypeptides (i.e. <10 residues); proteins such as human serum albumin, bovine serum albumin, gelatin or immunoglobulins; hydrophilic polymers such as polyvinylpyrrolidone; monosaccharides such as xylose, mannose, fructose and glucose; and polysaccharides such as dextran.

In certain embodiments, the concentration of one or more excipients in the composition described herein is about 0.001 to 5 weight percent, while in other embodiments; the concentration of one or more excipients is about 0.1 to 2 weight percent. Excipients are well known in the art and are manufactured by known methods and available from commercial suppliers.

In one embodiment, the aqueous composition of the present invention further comprises arginine as stabilizing agent, preferably with a concentration from 5 mM to 50 mM, preferably from 15 mM to 30 mM, more preferably from 20 mM to 30 mM, most preferably about 25 mM.

In some instances, arginine-free compositions might be preferred as arginine can cause serious side effects in some people. A severe allergic reaction, called anaphylaxis, can occur after arginine injections, as well as stomach discomfort, including nausea, stomach cramps or an increased number of stools. In one embodiment, the aqueous composition of the present invention is substantially free of arginine as a free amino acid. More preferably, the composition is substantially free of any free amino acids. As used herein, the term “substantially free” refers to a formulation containing less than 0.033%, less than 0.001%, less than 0.0005%, less than 0.0003%, or less than 0.0001% of arginine as a free amino acid (or preferably of any free amino acid). For example, the composition does not comprise arginine, preferably neither comprises arginine, nor cysteine, nor proline, nor glycine, nor methionine, nor histidine, nor serine, nor valine, nor lysine, nor glutamate. Note that although the composition according to the present invention does preferably not comprise arginine as a free amino acid (or, more preferably, any other amino acid such as proline, glycine, methionine, histidine, serine, valine, lysine, glutamate), the antibody itself can contain arginine (or any other amino acid) residues in its chain.

In one embodiment, the aqueous composition of the present invention is essentially free of one or more preservatives, such as benzyl alcohol, phenol, m-cresol, chlorobutanol and benzethonium Cl. In another embodiment, a preservative may be included in the formulation, particularly where the formulation is a multidose formulation. The concentration of preservative may be in the range from about 0.1% to about 2%, most preferably from about 0.5% to about 1%.

The particular embodiments of the invention described below, may optionally be combined with one or more of the features described above or below:

### Acetate Buffer/Dissacharide Formulations

In a particular embodiment, the composition of the invention comprises or consists of:
an anti-TNFα antibody;
an acetate buffer at a concentration from 10 mM to 50 mM; and
a disaccharide at a concentration of less than 140 mM; and
optionally, from 0.01% w/v to 1% w/v of a surfactant;
wherein the pH of the composition is from pH 4.0 to pH 7.0.

In one embodiment, the composition according to the above embodiment further comprises a surfactant at a concentration from 0.05% w/v to 0.2% w/v, preferably of about 0.1% w/v.

It is preferred that the anti-TNFα antibody is a human anti-hTNFα antibody, preferably the anti-TNFα antibody is adalimumab. The anti-TNFα antibody or human anti-hTNFα antibody or adalimumab concentration may be from 5 to 500 mg/mL, from 7.5 to 250 mg/mL, from 10 mg/ml to 200 mg/mL, from 10 mg/ml to 150 mg/mL or from 10 mg/ml to 100 mg/mL. In a particular embodiment, the anti-TNFα antibody concentration is from 40 to 200 mg/mL, such as 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195 or 200 mg/mL In addition, it is preferred that said composition further comprises a salt present at a concentration of more than 100 mM or from 80 to 130 mM, preferably wherein the salt concentration is 105 mM, more preferably wherein the salt is sodium chloride.

In a particular embodiment, the composition according to the present invention comprises, or preferably consists of, 10 mg/ml to 200 mg/ml (preferably 50 mg/ml) of adalimumab, from 10 mM to 50 mM of an acetate buffer, from 40 mM to 130 mM of a disaccharide, from 90 to 115 mM of a salt and optionally from 0.05% w/v to 0.2% w/v of a surfactant. Preferably, the pH of the composition is from pH 4.4 to pH 6.5, more preferably from pH 4.0 to pH 5.7, even more preferably from pH 4.4 to pH 5.2.

Preferably, said surfactant is polysorbate 80. It is also preferred that the disaccharide is trehalose, sucrose or a combination thereof. In a particular embodiment, when the disaccharide is trehalose, it is present at a concentration from 40 mM to 130 mM. In another particular embodiment, when the disaccharide is sucrose, it is present at a concentration from 80 mM to 120 mM, preferably at a concentration of 100 mM.

Preferably, said acetate buffer is selected from histidine acetate and sodium acetate.

In a preferred embodiment, the composition according to the present invention comprises, or preferably consists of, 10 mg/ml to 200 mg/ml (preferably 50 mg/ml) of adalimumab, from 10 mM to 50 mM histidine acetate, from 55 mM to 75 mM trehalose, from 90 to 115 mM NaCl and from 0.05% w/v to 0.2% w/v polysorbate 80. Preferably, the pH of the composition is from pH 4.7 to pH 5.7.

In a more preferred embodiment, the composition according to the present invention comprises, or preferably consists of, 10 mg/ml to 200 mg/ml (preferably 50 mg/ml) of adalimumab, 20 mM histidine acetate, 65 mM trehalose, 105 mM NaCl and 0.1% w/v polysorbate 80, wherein the pH of the composition is about pH 5.2.

In another preferred embodiment, the composition according to the present invention comprises, or preferably consists of, 10 mg/ml to 200 mg/ml (preferably 50 mg/ml) of adalimumab, from 10 mM to 50 mM histidine acetate, from 55 mM to 75 mM trehalose and from 90 to 115 mM NaCl. Preferably, the pH of the composition is from pH 4.7 to pH 5.7.

In a more preferred embodiment, the composition according to the present invention comprises, or preferably consists of, 10 mg/ml to 200 mg/ml (preferably 50 mg/ml) of adalimumab, 20 mM histidine acetate, 65 mM trehalose, and 105 mM NaCl, wherein the pH of the composition is about pH 5.2.

In another preferred embodiment, the composition according to the present invention comprises, or preferably consists of, 10 mg/ml to 200 mg/ml (preferably 50 mg/ml) of adalimumab, from 10 mM to 50 mM sodium acetate, from 80 mM to 120 mM sucrose, from 90 to 115 mM NaCl and optionally from 0.05% w/v to 0.2% w/v polysorbate 80. Preferably, the pH of the composition is from pH 4.7 to pH 5.7.

In a more preferred embodiment, the composition according to the present invention comprises, or preferably consists of, 10 mg/ml to 200 mg/ml (preferably 50 mg/ml) of adalimumab, 20 mM sodium acetate, 100 mM sucrose, 105 mM NaCl and 0.1% w/v polysorbate 80, wherein the pH of the composition is about pH 5.2.

In a further preferred embodiment, the composition according to the present invention comprises, or preferably consists of, 10 mg/ml to 200 mg/ml (preferably 50 mg/ml) of adalimumab, from 10 mM to 50 mM sodium acetate, from 80 mM to 120 mM sucrose, from 15 mM to 30 mM Arginine, from 90 to 115 mM NaCl and, optionally from 0.05% w/v to 0.2% w/v polysorbate 80. Preferably, the pH of the composition is from pH 4.7 to pH 5.7.

In a more preferred embodiment, the composition according to the present invention comprises, or preferably consists of, 10 mg/ml to 200 mg/ml (preferably 50 mg/ml) of adalimumab, 20 mM sodium acetate, 100 mM sucrose, 25 mM Arginine, 105 mM NaCl and 0.1% w/v polysorbate 80, wherein the pH of the composition is about pH 5.2.

In another more preferred embodiment, the composition according to the present invention comprises, or preferably consists of, 10 mg/ml to 200 mg/ml (preferably 50 mg/ml) of adalimumab, 20 mM sodium acetate, 100 mM sucrose, 25 mM Arginine, 105 mM NaCl, wherein the pH of the composition is about pH 5.2.

### Citrate or Citrate-Acetate Buffer/Sugar Alcohol Formulations

In another particular embodiment, the composition of the invention comprises or consists of:
an anti-TNFα antibody;
a citrate or citrate-acetate buffer;
a sugar alcohol at a concentration from 50 mM to 300 mM; and
optionally, a surfactant;
   wherein the pH of the composition is from pH 4.0 to pH 7.0.

Preferably, the composition of the invention comprises or consists of:
an anti-TNFα antibody;
a citrate or citrate-acetate buffer at a concentration from 10 mM to 50 mM;
a sugar alcohol at a concentration from 50 mM to 300 mM; and
optionally, from 0.01% w/v to 1% w/v of a surfactant;
wherein the pH of the composition is from pH 4.0 to pH 7.0.

In one embodiment, the composition according to the above embodiment further comprises a surfactant at a concentration from 0.05% w/v to 0.2% w/v, preferably of about 0.1% w/v.

It is preferred that the anti-TNFα antibody is a human anti-hTNFα antibody, preferably the anti-TNFα antibody is adalimumab. The anti-TNFα antibody or human anti-hTNFα antibody or adalimumab concentration may be from 5 to 500 mg/mL, from 7.5 to 250 mg/mL, from 10 mg/ml to 200 mg/mL, from 10 mg/ml to 150 mg/mL or from 10 mg/ml to 100 mg/mL. In a particular embodiment, the anti-TNFα antibody concentration is from 40 to 200 mg/mL, such as 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195 or 200 mg/mL In addition, it is preferred that said composition further comprises a salt present at a concentration of more than 100 mM or from 80 to 130 mM, preferably wherein the salt concentration is 105 mM, more preferably wherein the salt is sodium chloride.

In another particular embodiment, the composition according to the present invention comprises, or preferably consists of, 10 mg/ml to 200 mg/ml (preferably 50 mg/ml) of adalimumab, from 10 mM to 50 mM of a citrate or citrate-acetate buffer, from 50 to 300 mM sugar alcohol, from 90 to 115 mM salt and optionally from 0.05% w/v to 0.2% w/v of a surfactant. Preferably, the pH of the composition is from pH 4.0 to pH 5.7, more preferably from pH 4.4 to pH 5.2. It is also preferred that said sugar alcohol is mannitol, said surfactant is polysorbate 80, and/or said salt is sodium chloride (NaCl). Preferably, said composition is substantially free of arginine as a free amino acid.

In a preferred embodiment, the composition according to the present invention comprises, or preferably consists of, 10 mg/ml to 200 mg/ml (preferably 50 mg/ml) of adalimumab, 10 mM to 50 mM of a citrate or citrate-acetate buffer, from 50 to 300 mM mannitol, from 90 to 115 mM NaCl, and from 0.05% w/v to 0.2% w/v of polysorbate 80. Preferably, the pH of the composition is from pH 4.0 to pH 5.7, more preferably about pH 4.4 or about pH 5.2.

In another preferred embodiment, the composition according to the present invention comprises, or preferably consists of, 10 mg/ml to 200 mg/ml (preferably 50 mg/ml) of adalimumab, 10 mM to 50 mM of a citrate or citrate-acetate buffer, from 50 to 300 mM mannitol and from 90 to 115 mM NaCl. Preferably, the pH of the composition is from pH 4.0 to pH 5.7, more preferably about pH 4.4 or about pH 5.2.

Preferably, said buffer is sodium citrate or citrate-acetate buffer.

In a more preferred embodiment, the composition according to the present invention comprises, or preferably consists of, 10 mg/ml to 200 mg/ml (preferably 50 mg/ml) of adalimumab, 20 mM sodium citrate, 65 mM mannitol, 105 mM NaCl and 0.1% w/v of polysorbate 80, wherein the pH of the composition is about pH 5.2.

In an additional more preferred embodiment, the composition according to the present invention comprises, or preferably consists of, 10 mg/ml to 200 mg/ml (preferably 50 mg/ml) of adalimumab, 20 mM sodium citrate, 65 mM mannitol, and 105 mM NaCl, wherein the pH of the composition is about pH 5.2.

In another more preferred embodiment, the composition according to the present invention comprises, or preferably consists of, 10 mg/ml to 200 mg/ml (preferably 50 mg/ml) of adalimumab, 10 mM sodium citrate, 85 mM mannitol, 105 mM NaCl and 0.1% w/v of polysorbate 80, wherein the pH of the composition is about pH 5.2.

In a further more preferred embodiment, the composition according to the present invention comprises, or preferably consists of, 10 mg/ml to 200 mg/ml (preferably 50 mg/ml) of adalimumab, 20 mM sodium citrate, 65 mM mannitol, 105 mM NaCl and optionally 0.1% w/v of polysorbate 80, wherein the pH of the composition is about pH 4.4, preferably pH 4.42.

In an additional more preferred embodiment, the composition according to the present invention comprises, or preferably consists of, 10 mg/ml to 200 mg/ml (preferably 50 mg/ml) of adalimumab, 20 mM citrate-acetate, 65 mM mannitol, 105 mM NaCl and 0.1% w/v polysorbate 80, wherein the pH of the composition is about pH 5.2.

In another additional more preferred embodiment, the composition according to the present invention comprises, or preferably consists of, 10 mg/ml to 200 mg/ml (preferably 50 mg/ml) of adalimumab, 20 mM citrate-acetate, 65 mM mannitol, and 105 mM NaCl, wherein the pH of the composition is about pH 5.2.

In an additional particular embodiment, the composition according to the present invention comprises, or preferably consists of, 10 mg/ml to 200 mg/ml (preferably 50 mg/ml) of adalimumab, from 10 mM to 50 mM a citrate or citrate-acetate buffer, from 50 to 300 mM a sugar alcohol and optionally from 0.05% w/v to 0.2% w/v of a surfactant. Preferably, the pH of the composition is from pH 4.0 to pH 5.7.

In a preferred embodiment, the composition according to the present invention comprises, or preferably consists of, 10 mg/ml to 200 mg/ml (preferably 50 mg/ml) adalimumab, 10 mM to 50 mM of sodium citrate buffer, from 50 to 300 mM mannitol and optionally from 0.05% w/v to 0.2% w/v of polysorbate 80. Preferably, the pH of the composition is from pH 4.0 to pH 5.7.

In a more preferred embodiment, the composition according to the present invention comprises, or preferably consists of, 10 mg/ml to 200 mg/ml (preferably 50 mg/ml) of adalimumab, 20 mM sodium citrate, 275 mM mannitol, and 0.1% w/v of polysorbate 80, wherein the pH of the composition is about pH 5.2.

In an even further particular embodiment, the composition according to the present invention comprises, or preferably consists of, 10 mg/ml to 200 mg/ml (preferably 50 mg/ml) of adalimumab, 10 mM to 50 mM of a phosphate buffer, from 50 to 100 mM of a sugar alcohol, from 90 to 115 mM of a salt, and optionally from 0.05% w/v to 0.2% w/v of a surfactant. Preferably, the pH of the composition is from pH 5.5 to pH 6.5.

In a preferred embodiment, the composition according to the present invention comprises, or preferably consists of, 10 mg/ml to 200 mg/ml (preferably 50 mg/ml) of adalimumab, 10 mM to 50 mM of sodium phosphate buffer, from 50 to 100 mM mannitol, from 90 to 115 mM NaCl, and optionally from 0.05% w/v to 0.2% w/v polysorbate 80. Preferably, the pH of the composition is from pH 5.5 to pH 6.5.

In a more preferred embodiment, the composition according to the present invention comprises, or preferably consists of, 10 mg/ml to 200 mg/ml (preferably 50 mg/ml) of adalimumab, 20 mM sodium phosphate, 65 mM mannitol, 105 mM NaCl and 0.1% w/v polysorbate 80, wherein the pH of the composition is about pH 6.0.

### Methods of Treatment

The composition of the invention may suitable for in vitro or in vivo use. Preferably, said composition is a pharmaceutical composition. In a second aspect, the present invention relates to the use of the aqueous composition of the invention as a pharmaceutical composition. It also relates to the composition or pharmaceutical composition of the invention for use as a medicament or for use in therapy.

As used herein, a “pharmaceutical composition” refers to a composition that is pharmaceutically acceptable. The term “pharmaceutically acceptable” as used herein refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio.

Said pharmaceutical composition of the invention is formulated to be compatible with its intended route of administration. Methods to accomplish the administration are known to those of ordinary skill in the art. This includes, for example, injections, by parenteral routes such as intravenous, intravascular, intraarterial, subcutaneous, intramuscular, intraperitoneal, intraventricular, intraepidural, or others as well as oral, nasal, ophthalmic, rectal, or topical. Sustained release administration is also specifically contemplated, by such means as depot injections or erodible implants.

In one embodiment, the aqueous composition is administered to a subject by subcutaneous administration. In a more preferred embodiment, said pharmaceutical composition of the invention is for subcutaneous injection. For such purposes, the composition may be injected using a syringe, as well as other devices including injection devices (e.g., the Inject-ease and Genject devices); injector pens (such as the GenPen); needleless devices (e.g., MediJector and Biojectorr 2000); and subcutaneous patch delivery systems. In one embodiment, the device, e.g., a syringe, autoinjector pen, contains a needle with a gauge ranging in size from 25 G or smaller in diameter. In one embodiment, the needle gauge ranges in size from 25 G to 33 G (including ranges intermediate thereto, e.g., 25 sG, 26, 26 sG, 27 G, 28 G, 29 G, 30 G, 31 G, 32 G, and 33 G). In a preferred embodiment, the smallest needle diameter and appropriate length is chosen in accordance with the viscosity characteristics of the formulation and the device used to deliver the composition of the invention.

The invention also includes methods for delivery to a subject using a needleless device. Such a device allows for dispersion of the protein throughout the tissue of a subject without the need for an injection by a needle. Examples of needleless devices include, but are not limited to, Biojectorr 2000 (Bioject Medical Technologies), Cool.Click (Bioject Medical Technologies), Iject (Bioject Medical Technologies), Vitajet 3, (Bioject Medical Technologies), Mhi500 (The Medical House PLC), Injex 30 (INJEX-Equidyne Systems), Injex 50 (INJEX-Equidyne Systems), Injex 100 (INJEX-Equidyne Systems), Jet Syringe (INJEX-Equidyne Systems), Jetinjector (Becton-Dickinson), J-Tip (National Medical Devices, Inc.), Medi-Jector VISION (Antares Pharma), MED-JET (MIT Canada, Inc.), DermoJet (Akra Dermojet), Sonoprep (Sontra Medical Corp.), PenJet (PenJet Corp.), MicroPor (Altea Therapeutics), Zeneo (Crossject Medical Technology), Mini-Ject (Valeritas Inc.), ImplaJect (Caretek Medical LTD), Intraject (Aradigm), and Serojet (Bioject Medical Technologies).

Also included in the invention are delivery devices that house the aqueous composition of the invention. Examples of such devices include, but are not limited to, a syringe, a pen (such as an autoinjector pen), an implant, an inhalation device, a needleless device, and a patch. In addition, the invention includes methods of delivering the compositions of the invention by inhalation and inhalation devices containing said composition for such delivery. In one embodiment, the aqueous formulation is administered to a subject via inhalation using a nebulizer or liquid inhaler. Generally, nebulizers use compressed air to deliver medicine as wet aerosol or mist for inhalation, and, therefore, require that the drug be soluble in water. Types of nebulizers include jet nebulizers (air-jet nebulizers and liquid-jet nebulizers) and ultrasonic nebulizers.

In a third aspect, the invention relates to the composition or pharmaceutical composition as described in the preceding aspects for use in a method for treating inflammatory and/or immune system mediated diseases, more specifically for use in a method for treating inflammatory and/or immune system mediated diseases associated with an increase of TNFα. A related aspect, is directed to methods of treating inflammatory and/or immune system mediated diseases, more specifically to methods of treating inflammatory and/or immune system mediated diseases associated with an increase of TNFα, with a composition or pharmaceutical composition of the present invention. In one embodiment, the method to treat said inflammatory and/or immune system mediated diseases, more specifically inflammatory and/or immune system mediated diseases associated with an increase of TNFα, comprises administering to a subject a composition or pharmaceutical composition of the present invention in an amount effective to treat said inflammatory and/or immune system mediated diseases.

The therapeutic effect of the anti-TNFα antibody comprised in the compositions according to the present invention is known in the art and includes, but is not limited thereto, treating rheumatoid arthritis, polyarticular juvenile idiopathic arthritis, psoriatic arthritis, ankylosing spondylitis, granulomatosis, Crohn's disease, ulcerative colitis, chronic obstructive pulmonary disease, hepatitis C, endometriosis, asthma, cachexia, psoriasis, hidradenitis suppurativa or atopic dermatitis, or other inflammatory or immune system mediated disease, disorder, or condition, more specifically when this disease, disorder or condition is associated to an increase of TNFα. The compositions may be administered in an amount sufficient to treat (alleviate symptoms, halt or slow progression of) the disorder (e.g., a therapeutically effective amount).

The term “treating”, as used herein, unless otherwise indicated, includes the amelioration, cure, and/or maintenance of a cure (i.e., the prevention or delay of relapse) of a disease or disorder. Treatment after a disorder has started aims to reduce, alleviate, ameliorate or altogether eliminate the disorder, and/or its associated symptoms, to prevent it from becoming worse, to slow the rate of progression, or to prevent the disorder from re-occurring once it has been initially eliminated (i.e., to prevent a relapse).

The term “treatment”, as used herein, unless otherwise indicated, refers to the act of “treating” defined immediately above.

As used herein, a “therapeutically effective amount” of an anti-TNFα antibody may refer to an amount of an antibody that is effective, upon single or multiple dose administration to a subject (such as a human patient) at treating inflammatory or immune system mediated disease, more specifically at treating inflammatory or immune system mediated disease associated to an increase of TNFα.

In an additional aspect, the invention relates to a method of manufacturing an aqueous composition according to the preceding aspects which comprises the steps of:
preparing a buffer selected from the list consisting of an acetate buffer, a citrate buffer, or a citrate-acetate buffer at the required pH,
adding a disaccharide and/or sugar alcohol, and optionally, a surfactant and/or salt,
adding an aqueous solution to the final volume and when necessary adjusting the pH,
incorporating an anti-TNFα antibody to the composition.

Said aqueous solution added to complete the final volume is preferably water. Typically, deionized water is used, preferably ‘ultrapure’ water of “Type 1”, as defined by various authorities (e.g. ISO 3696), such as milliQ water.

The anti-TNFα antibody may be found in purified form or in a solution, e.g. a buffer. Where the antibody is found in a solution, the incorporation of the antibody into the aqueous composition is usually performed by replacing the solution by dialysis (i.e., buffer exchange by dialysis).

The term “dialysis” as used herein refers to the diffusion of dissolved solutes across a selectively permeable membrane against a concentration gradient in an effort to achieve equilibrium. While small solutes pass through the membrane larger solutes are trapped on one side. By exchanging the dialysate buffer on the outside side of the membrane, you can continually pull away the smaller solutes to purify the trapped larger molecules.

Several rounds of dialysis can be used for buffer exchange. In general, dialysis will be most effective when the buffer is replaced a few times, for example 2, or 3 times, and then preferably left overnight at room temperature on a stir plate. A standard protocol for dialysis is 16 to 24 hours. Many factors affect the dialysis rate, including: diffusion coefficients, pH, temperature, time, concentration of species, sample volume, dialysate (buffer) volume, number of dialysate changes, membrane surface area, membrane thickness, molecular charges and dialysate agitation (stirring).

Several types of membranes for dialysis are commercially available and are well known in the art. Illustrative non limiting examples are Polyvinylidene Difluoride (PVDF) membranes, cellulose ester (CE) membranes and regenerated cellulose (RC) membranes.

It is contemplated that any embodiment discussed in this specification can be implemented with respect to any composition, pharmaceutical composition, medical use, method of treatment, and method of manufacturing said composition; and vice versa. It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” may mean “one,” but it is also consistent with the meaning of “one or more,” “at least one,” and “one or more than one”. The use of the term “another” may also refer to one or more. The use of the term “or” in the claims is used to mean “and/or” unless explicitly indicated to refer to alternatives only or the alternatives are mutually exclusive.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. As used herein, the phrase “consisting essentially of” limits the scope of a claim to the specified materials or steps and those that do not materially affect the basic and novel characteristic(s) of the claimed invention. As used herein, the phrase “consisting of excludes any element, step, or ingredient not specified in the claim except for, e.g., impurities ordinarily associated with the element or limitation.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

The following examples serve to illustrate the present invention and should not be construed as limiting the scope thereof.

### EXAMPLES

### Materials and Methods

### Analytical Methods

### 1.—Visual Inspection

For visual inspection the vials were inspected for the presence or absence of visible particles under gentle, manual, radial agitation for 5 seconds in front of a white background and for 5 seconds in front of a black background according to the European Pharmacopoeia (7th edition; monograph 2.9.20). The inspection was performed by two independent, trained examiners.

2.—pH

Formulation pH was measured with a calibrated pH meter (SevenEasy®, Mettler Toledo AG, Schwerzenbach, Switzerland) using normal ionic strength electrode (InLab® Micro).

### 3.—Osmolality

Osmolality of the samples was measured by method of freezing-point depression using a Knauer Automatic Semi-Micro Osmometer K-7400 (Knauer, Berlin, Germany).

### 4.—UV Spectroscopy

UV absorbance was measured by using an Agilent 8453 UV spectrophotometer. The analysis was performed in a 0.01-cm pathlength quartz cuvette. The samples were measured undiluted at 50 mg/ml. The concentration of mAb was calculated using an extinction coefficient A280 nm=1.39 ml mg-1 cm-1. The optical density determined at 350 nm was used to assess the turbidity and the extent of light scattering in the sample, respectively, which may be expressed as the aggregation index A.I.: A.I.=100*(A350/(A280−A350)). The absorbance at 350 nm as well as A.I. are sensitive to insoluble aggregates, but less sensitive to low levels of soluble aggregates in the solutions. As control material bovine serum albumin (BSA) at a concentration of 2 mg/ml was used (extinction coefficient A280 nm=0.667 (ml/mg)-1(cm-1).

### 5.—Micro-Flow Imaging (MFI)

The Micro-Flow Imaging measurements were conducted at a DPA-5200 particle analyzer system (ProteinSimple, Santa Clara, Calif., USA) equipped with a silane coated high-resolution 100 μm flow cell. The samples were analyzed at a concentration of 5 mg/ml, diluted in placebo formulation. A pre-run volume of 0.17 ml was followed by a sample run of 0.28 ml. Approximately 1100 images were taken per sample. Between the measurements, the flow cell was cleaned with purified water and the background illumination was optimized using placebo formulation. MFI View System Software (MVSS) version 2-R2-6.1.20.1915 was used to perform the measurements and MFI View Analysis Suite (MVAS) software version 1.3.0.1007 was used to analyze the samples.

### 6.—Resonant Mass Measurement (ARCHIMEDES)

RMM was performed by using the ARCHIMEDES particle metrology system (Affinity Biosensors, Santa Barbara, Calif., USA) equipped with a Micro sensor (size range 0.3-4 μm) calibrated with 1 μm polystyrene standards.

All samples were analyzed at a concentration of 5 mg/ml, diluted in placebo formulation. Some samples had to be diluted 2-fold with MQ water to obtain reliable data. Before measurement, the system was filled with sample and the lower size limit of detection (LOD) was determined three times in the automatic mode and the mean value was set as fixed limit of detection for the measurement. The LOD correspond the lowest detectable particle size within one sample. For negatively buoyant particles (proteinaceous particles) the LOD is around 0.3 μm depending on the noise level within a sample, so that particle sizes ≧0.3 μm can be feasibly characterized. For positively buoyant particles (silicone oil-like droplets), the LOD is slightly higher and usually at about 0.5 μm (due to the smaller difference in particle density relative to buffer). For samples that have a higher LOD than 0.3 μm or 0.5 μm the actual total particle concentration might be higher than reported.

The buffer density was determined individually for each formulation buffer and particle density was arbitrarily set to 1.32 g/ml for negatively buoyant particles (proteinaceous particles) and 0.97 g/ml for positively buoyant particles (silicone oil-like droplets) based on the recommendations of the manufacturer. The measurement stop trigger was set to a volume of 0.15 μl and the overall sample volume for triplicate measurement was 600 μl. Between each measurement the system was rinsed with ultra-pure water. Results were analyzed using the ParticleLab software (v1.9.50) with a size bin step of 10 nm.

The lower limit of quantification (LOQ) of total particles (i.e. particle size class ≧0.3 μm) recommended by the manufacturer is 300,000 particles per ml (the minimal number of particles detected in the small volume analyzed, i.e. 0.15 μl, must be >50 particles). Total particle concentrations of below 300,000 particles per ml should not be considered statistically relevant.

### 7.—Dynamic Light Scattering (DLS)

DLS measurements were carried out by using a Zetasizer APS 2000 plate reader (Malvern Instruments, Worcestershire, UK) instrument. For each sample, 100 μl were analyzed in a Corning 96-well plate at 20° C. All samples were analyzed at a concentration of 5 mg/ml, diluted in placebo formulation.

The automatic measurement mode of the software was used to allow optimum measurement settings, such as measurement time and number of acquisitions (typically 10-15 acquisitions of each 10 seconds). Furthermore, instrument parameters such as attenuator setting were optimized by the software to obtain the recommended count rates. Each sample was measured three times.

The Malvern Zetasizer software (Version 7.03) was used to calculate Z-average diameter, polydispersity index (PdI) and particle size distribution (by intensity) using the “protein analysis” algorithm. From the software's database, protein material (RI=1.450, absorption=0.001) and water as solvent (RI=1.330, viscosity=1.0031 cP at 20.0° C.) were selected.

8.—Microcalorimetry (g-DSC)

The MicroCal VP-Capillary DSC System (GE Healthcare) was used for ultrasensitive calorimetric analysis of the protein formulations to determine the melting temperature (Tm) of the protein.

For the measurements about 0.4 ml diluted protein formulation was required. The concentrated mAb samples were diluted 25-fold with the corresponding formulation buffer to achieve a target concentration of ˜2 mg/ml mAb. To improve the normalization of the g-DSC data by eliminating concentration differences, the protein concentration was determined via UV absorption measurements at 280 nm (A280 nm) after dilution. A Tecan Safire2 plate reader (Tecan Austria GmbH, Grödig, Austria) was used and a triplicate of 200 μl of the sample was measured in a Costar UV 96-well plate (Corning Incorporation, NY, USA). The absorbance of the corresponding formulation buffer was subtracted. The specific extinction coefficient of 1.39 ml/mg was used to calculate the protein concentration. In addition, a molecular weight of 148 kDa was used to normalize the obtained heat capacity (Cp) to molar values. The samples were cooled at 5° C. in the autosampler system before analysis. To determine the background, reference and sample cell were filled with formulation buffer and scanned from 10 to 100° C. with 60° C./h. To determine the Tm of the protein, the sample cell was filled with the diluted protein solution and the reference cell with formulation buffer, and one heating scan from 10 to 100° C. at 60° C./h was performed.

After the protein scan, the cells were filled with 10% Decon 90 solution and scanned with the same settings of the protein sample. Subsequently, the cells were washed extensively with highly purified water to remove the detergent.

The background obtained from the corresponding formulation buffer scan was subtracted from the sample measurement. The Tm values were determined from the peak maxima of the deconvoluted unfolding transitions after baseline subtraction (non-two state model with 4 transitions). Data analysis was performed by using Origin 7.0 DSC software.

### 9.—High Performance Size-Exclusion Chromatography (HP-SEC)

The following parameters were used for the HP-SEC analysis:
Instrument: HP1200 (Agilent Technology)
Column: Super SW3000; 4.6×300 mm (4 μm); TOSOH Bioscience, Cat. No.: 18675.
lot number 95W
Flow rate: 0.35 ml/min Mobile phase: 0.2 M sodium phosphate, pH 6.8±0.2
Detection: UV at 280 nm, reference off
Bandwidth: 4 nm, reference off Column oven: 30° C. Sample cooling: 4° C. Injection: draw speed and eject speed 450 μl/min Injection volume: 30 μg (30 μl for 1.0 mg/ml sample)
Analysis time: 20 min

### 10.—Ion-Exchange Chromatography (IEX)

The following parameters were used for the IEX analysis:
Instrument: HP1200 (Agilent Technology)
Column: Dionex Propac WCX-10, 4.0 mm d.i.×250 mm (weak cation exchange resin) CV=3.14 ml; Dionex Cat. No.: 054993.
lot number 014-27-012
Mobile phases: Mobile phase A: 10 mM sodium phosphate (pH 7.40±0.05)
Mobile phase B: 10 mM sodium phosphate, 0.25 M NaCl (pH 7.40±0.05)
Flow rate: 0.5 ml/min
Detection: UV at 280 nm, reference off
*214 nm, reference 360 nm
Bandwidth: 16 nm, reference off (280 nm)
*4 nm, reference 100 nm (214 nm)
Column oven: 35° C. Sample cooling: 4° C.
Injection: draw speed and eject speed 450 μL/min Injection volume: 40 μg (20 μL for 2 mg/mL sample)
Analysis time: 40 min.

### 11.—SDS-PAGE Reduced and Non-Reduced Electrophoresis

The purpose of SDS-PAGE electrophoresis is to separate proteins according to their size, and no other physical features, while in the non-reduced gel electrophoresis the proteins are prepared in non reducing, non denaturing sample buffer and electrophoresis is also performed in the absence of denaturing and reducing agents. Following electrophoresis, the gel was stained with Coomassie Brilliant Blue R-250 allowing visualization of the separated proteins.

The analytical methods used herein may have the purpose indicated in the table below:

| Method | Purpose |
|---|---|
| μDSC | Melting temperature |
| Dynamic light | Particle size distribution in the nm-size range |
| scattering (DLS) | (1 to 1000 nm) |
| HP-SEC | Purity, monomer content, aggregates, fragments |
| IEX-HPLC | Charge variants |
| Micro-Flow | Quantification and visualization of subvisible |
| Imaging (MFI) | particles (~1-100 μm) Differentiation silicone |
| | oil from proteinaceous particles |
| Osmolality | Osmolality of formulations |
| pH | Formulation pH |
| Resonant mass | Quantification of subvisible particles (~300 nm |
| measurements (RMM) | to 5 μm) Differentiation silicone oil from |
| | proteinaceous particles |
| UV spectroscopy | Protein content (A280) and turbidity |
| Visual inspection | Visible particles |
| SDS-PAGE reduced | Purity, monomer content, aggregates, fragments |
| and non-reduced | |

### Example 1

### Sample Preparation

For the studies described in Example 1, HUMIRA® Drug Product (formulated in 6-14 mM sodium citrate/phosphate, 65 mM mannitol, 105 mM NaCl, 0.1% PS80, pH 5.2 (i.e., F1 or Innovator formulation) was used as starting material. Buffer exchange was performed by dialysis.

### Formulations

The following adalimumab samples in the following buffers were analyzed:

Formulation components

Humira formulation (Innovator reference formulation)
F1. Same buffer as Innovator Formulation (sodium chloride, monobasic
sodium phosphate dihydrate, dibasic sodium phosphate dihydrate, sodium
citrate, citric acid monohydrate) pH 5.2, 65 mM mannitol, 0.10% PS 80
F2. 20 mM Na Citrate (Na citrate dihydrate, citric acid, monohydrate),
pH 5.2. 65 mM mannitol, 105 mM NaCl, 0.10% PS80
F3. 20 mM Na phosphate (Na phosphate monobasic, monohydrate, Na
phosphate dibasic, anhydrous), pH 6.0, 65 mM mannitol, 105 mM NaCl,
0.10% PS80
F4. 20 mM Histidine acetate (L-Histidine and acetic acid), pH 5.2,
65 mM trehalose, 105 mM NaCl, 0.10% PS80
F8. 20 mM Na acetate (Na acetate anhydrous and acetic acid), pH
5.2, 100 mM sucrose, 105 mM NaCl, 0.10% PS80
F9. 20 mM Na acetate (Na acetate anhydrous and acetic acid), pH
5.2, 100 mM sucrose, 105 mM NaCl, 25 mM Arginine (L-Arginine HCl),
0.10% PS80

Each sample comprises a nominal (target) concentration of 10 mg/ml of adalimumab. Placebo samples were also prepared without adalimumab antibody. The buffer of each sample was prepared as indicated below:
Buffer F1. Citrate-Phosphate, pH 5.2

| | | | | | Man | NaCl | PS80 | |
|---|---|---|---|---|---|---|---|---|
| pH | NaH2PO4*2H2O (g) | Na2HPO4*2H2O (g) | Na Citrate*2H2O (g) | Citric acid*H2O (g) | (g) | (g) | (g) | Water |
| 5.2 | 0.862 | 1.525 | 0.300 | 1.300 | 12.000 | 6.136 | 1.000 | to 1 L |

Buffer F2. Citrate, pH 5.2

| | Na | Citric | Man | NaCl | PS80 | |
|---|---|---|---|---|---|---|
| pH | Citrate*2H2O (g) | acid*H2O (g) | (g) | (g) | (g) | Water |
| 5.2 | 5.8824 g/L × | 4.2028 g/L × | 11.840 | 6.136 | 1.000 | to 1 L |
| | A (L) | B (L) | | | | |

20 mM Na Citrate, pH 5.2:
A: 20 mM Na citrate*2H2O (5.8824 g/L)
B: 20 mM citric acid*H2O (4.2028 g/L)
Add B over A to reach pH 5.2
Add soluble components Mannitol, NaCl and PS80. Add milli-Q water to the final volume. Adjust pH to 5.2, when necessary.
Buffer F3. Phosphate, pH 6.0

| | | Na2HPO4 | Man | NaCl | PS80 | |
|---|---|---|---|---|---|---|
| pH | NaH2PO4*H2O (g) | (g) | (g) | (g) | (g) | Water |
| 6.0 | 2.7598 g/L × | 2.8392 g/L × | 12.000 | 6.136 | 1.000 | to 1 L |
| | A (L) | B (L) | | | | |

20 mM Na phosphate, pH 6.0:
A: 20 mM NaH2PO4*H2O (2.7598 g/L)
B: 20 mM Na2HPO4 (2.8392 g/L)
Add 123 mL B over 877 mL A. pH 6.0
Add soluble components Mannitol, NaCl and PS80. Add milli-Q water to the final volume. Adjust pH to 6.0, when necessary.
Buffer F4. Histidine acetate, pH 5.2

| | Acetic acid | L-Histidine | TH | NaCl | PS80 | |
|---|---|---|---|---|---|---|
| pH | (g) | (g) | (g) | (g) | (g) | Water |
| 5.2 | 1.2010 g/L × | 3.1032 g/L × B (L) | 22.25 | 6.136 | 1.000 | to 1 L |
| | A (L) | | | | | |

20 mM Histidine acetate, pH 5.2:
A: Acetic acid 20 mM (1.20 g/L). pH 3.
B: L-Histidine 20 mM (3.10 g/L)
Add B over A. pH 5.2
Add soluble components L-Histidine, Trehalose, NaCl and PS80. Add milli-Q water to the final volume. Adjust pH to 5.2, when necessary.
Buffer F8. Acetate, pH 5.2

| | Acetic acid | Na Acetate | Suc | NaCl | PS80 | |
|---|---|---|---|---|---|---|
| pH | (g) | (g) | (g) | (g) | (g) | Water |
| 5.2 | 1.2010 g/L × | 1.6406 g/L × B (L) | 34.23 | 6.136 | 1.000 | to 1 L |
| | A (L) | | | | | |

20 mM Na acetate, pH 5.2:
A: Acetic acid 20 mM (1.20 g/L)
B: Na acetate 20 mM (1.64 g/L)
Add 643 mL B over 357 mL A. pH 5.2
Add soluble components Sucrose, NaCl and PS80. Add milli-Q water to the final volume. Adjust pH to 6.0, when necessary.
Buffer F9. Acetate-Arginine, pH 5.2

| | | Na | L- | | | | |
|---|---|---|---|---|---|---|---|
| | Acetic | Acetate | Arginine | Suc | NaCl | PS80 | |
| pH | acid (g) | (g) | (g) | (g) | (g) | (g) | Water |
| 5.2 | 1.2010 g/L × | 1.6406 g/ | 4.355 | 34.23 | 6.136 | 1.000 | to 1 L |
| | A (L) | L × B (L) | | | | | |

20 mM Na acetate, pH 5.2:
A: Acetic acid 20 mM (1.20 g/L)
B: Na acetate 20 mM (1.64 g/L)
Add 643 mL B over 357 mL A. pH 5.2
Add soluble components L-Arginine, Sucrose, NaCl and PS80. Add milli-Q water to the final volume. Adjust pH to 5.2 with HCl.

The following analyses were performed on the samples:
Visual appearance
pH
Absorbance 280 nm (protein concentration)
Absorbance 330 nm (turbidity)
DLS (sub-visible particle analysis)
Near-UV (Abs 250-350) spectroscopy (tertiary structure)
Size Exclusion HPLC Chromatography (SEC-HPLC)
SDS-PAGE reduced and non-reduced
Ion Exchange HPLC (IEX-HPLC)

### Stability Conditions.

The samples were subject to the following conditions:

| Protein | | |
|---|---|---|
| Conc. | | |
| (mg/mL) | Condition | Timepoints |
| 10 | t = 0 control | 1 |
| 10 | 2-8° C. storage (14 days) | 1 |
| 10 | 25° C. storage (14 days) | 1 |
| 10 | 40° C. storage (3, 7, 14 days) | 3 |
| 10 | 3 cycle FzTh (−80° C./20° C.) | 1 |
| 10 | Shake study 3 day at RT | 1 |

The freeze-thawing stress test (FzTh) consisted of: freeze 1 h −80° C., defrost 1 h +20° C., freeze 1 h −80° C., defrost 1 h +20° C., freeze 1 h −80° C., defrost 1 h +20° C.

The shake study test consisted of: Shaking at 400 rpm (horizontal shaking) for 72 hours at room temperature: visual inspection will be performed after 24, 48 and 72 hours.

At each time point pull, visual appearance was inspected to record color, clarity and presence of particulates, in real time. Vials were frozen at −80° C. until ready for analysis.

### Results

### 1. Visual Appearance

| | | | | | | | FzTh | Shake |
|---|---|---|---|---|---|---|---|---|
| | t = 0 control | 2-8° C. | 25° C. | 40° C. storage | | | 3 cycles | 3 day, RT |
| Cond. | (F1 to F9) | (F1 to F9) | (F1 to F9) | (F1 to F9, x3) | | | (F1 to F9) | (F1 to F9) |
| t = 0 | clear, no | clear, no | clear, no | clear, no | clear, no | clear, no | clear, no | clear, no |
| | color, no | color, no | color, no | color, no | color, no | color, no | color, no | color, no |
| | particulates | particulates | particulates | particulates | particulates | particulates | particulates | particulates |
| 3 d | | | | clear, no | | | | |
| | | | | color, no | | | | |
| | | | | particulates | | | | |
| 7 d | | | | | clear, no | | | |
| | | | | | color, no | | | |
| | | | | | particulates | | | |
| 14 d | | clear, no | clear, no | | | clear, no | | |
| | | color, no | color, no | | | color, no | | |
| | | particulates | particulates | | | particulates | | |
| 3x | | | | | | | clear, no | |
| FzTh | | | | | | | color, no | |
| | | | | | | | particulates | |
| Shake | | | | | | | | clear, no |
| study | | | | | | | | color, no |
| | | | | | | | | particulates |

All formulations at all timepoints and conditions presented as clear, no color and no visible particulates upon visual inspection and as compared to Innovator formulation.

### 2. pH Test

| | F1 | F2 | F3 | F4 | F8 | F9 |
|---|---|---|---|---|---|---|
| | PLACEBO | PLACEBO | PLACEBO | PLACEBO | PLACEBO | PLACEBO |
| t = 0 | 5.26 | 5.23 | 6.08 | 5.23 | 5.23 | 5.25 |

pH is measured to be close to target at the start of the study.

### 3. Protein Concentration by A280 nm

| | | F1 | F2 | F3 | F4 | F8 | F9 |
|---|---|---|---|---|---|---|---|
| t = 0 | Absorbance | 0.414 | 0.491 | 0.456 | 0.446 | 0.485 | 0.492 |
| | reading | | | | | | |
| | Protein | 9.64 | 11.43 | 10.60 | 10.37 | 11.29 | 11.47 |
| | concentration | | | | | | |
| | (mg/mL) | | | | | | |
| 4° C. 14 d | Absorbance | 0.458 | 0.476 | 0.411 | 0.417 | 0.463 | 0.453 |
| | reading | | | | | | |
| | Protein | 10.66 | 11.08 | 9.55 | 9.70 | 10.77 | 10.55 |
| | concentration | | | | | | |
| | (mg/mL) | | | | | | |
| 25° C. | Absorbance | 0.533 | 0.464 | 0.449 | 0.386 | 0.474 | 0.508 |
| 14 d | reading | | | | | | |
| | Protein | 12.41 | 10.78 | 10.43 | 8.99 | 11.02 | 11.84 |
| | concentration | | | | | | |
| | (mg/mL) | | | | | | |
| 40° C. | Absorbance | 0.528 | 0.499 | 0.621 | 0.471 | 0.594 | 0.509 |
| 14 d | reading | | | | | | |
| | Protein | 12.29 | 11.62 | 14.45 | 10.97 | 13.84 | 11.87 |
| | concentration. | | | | | | |
| | (mg/mL) | | | | | | |
| 3x FzTh | Absorbance | 0.496 | 0.454 | 0.433 | 0.426 | 0.460 | 0.473 |
| | reading | | | | | | |
| | Protein | 11.57 | 10.60 | 10.10 | 9.95 | 10.73 | 11.03 |
| | concentration | | | | | | |
| | (mg/mL) | | | | | | |
| Shake | Absorbance | 0.529 | 0.446 | 0.434 | 0.432 | 0.436 | 0.436 |
| study | reading | | | | | | |
| | Protein | 12.35 | 10.42 | 10.12 | 10.08 | 10.17 | 11.57 |
| | concentration | | | | | | |
| | (mg/mL) | | | | | | |

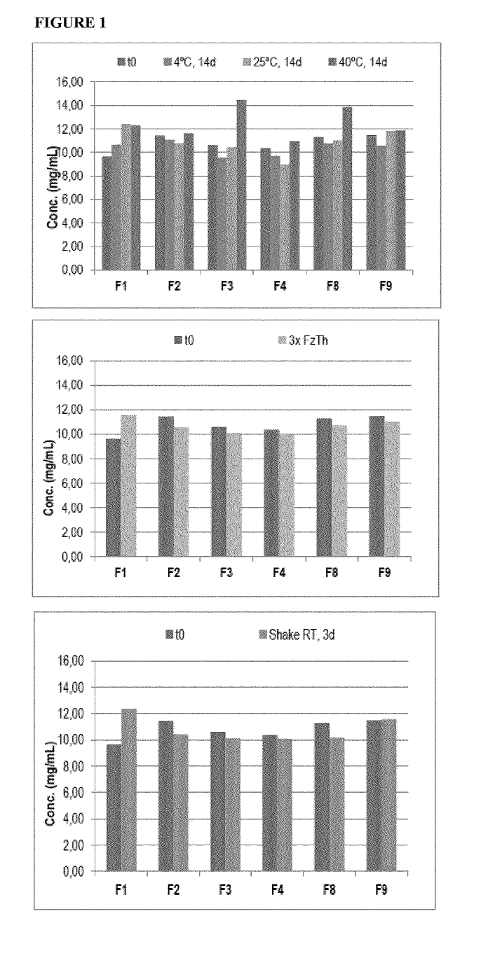

Intrinsic stability of Adalimumab was maintained when exchanged into the alternative buffer and excipient systems selected. Protein concentration remains consistent for all formulations at all conditions, as shown in FIG. 1.

### 4. Sub-Visible Particulate Analysis by DLS

| | | F1 | F2 | F3 | F4 | F8 | F9 |
|---|---|---|---|---|---|---|---|
| t = 0 | Size peak 1 | 12.34 | 12.44 | 12.61 | 12.14 | 13.14 | 13.84 |
| | (nm) | | | | | | |
| | Intensity | 100.0 | 100.0 | 100.0 | 100.0 | 100.0 | 100.0 |
| | Pdl | 0.117 | 0.085 | 0.118 | 0.034 | 0.052 | 0.063 |
| | (propositional | | | | | | |
| | dynamic logic) | | | | | | |
| 4° C. 14 d | Size peak 1 | 12.21 | 12.39 | 12.71 | 12.39 | 14.24 | 15.81 |
| | (nm) | | | | | | |
| | Intensity | 100.0 | 100.0 | 100.0 | 100.0 | 100.0 | 100.0 |
| | Pdl | 0.035 | 0.060 | 0.144 | 0.130 | 0.146 | 0.162 |
| 25° C. | Size peak 1 | 12.28 | 12.67 | 13.59 | 12.44 | 13.78 | 15.08 |
| 14 d | (nm) | | | | | | |
| | Intensity | 100.0 | 100.0 | 92.6 | 100.0 | 100.0 | 100.0 |
| | Size peak 2 | | | 118.1 | | | |
| | (nm) | | | | | | |
| | Intensity | | | 7.4 | | | |
| | Pdl | 0.118 | 0.061 | 0.169 | 0.091 | 0.103 | 0.103 |
| 40° C. | Size peak 1 | 12.35 | 12.37 | 13.05 | 12.84 | 13.11 | 17.02 |
| 14 d | (nm) | | | | | | |
| | Intensity | 100.0 | 100.0 | 91.8 | 90.6 | 100.0 | 100.0 |
| | Size peak 2 | | | 73.48 | 111.1 | | |
| | (nm) | | | | | | |
| | Intensity | | | 8.2 | 9.4 | | |
| | Pdl | 0.083 | 0.068 | 0.173 | 0.164 | 0.050 | 0.181 |
| 40° C. 3 d | Size peak 1 | 12.82 | 12.53 | 12.31 | 12.29 | 12.97 | 13.98 |
| | (nm) | | | | | | |
| | Intensity | 100.0 | 100.0 | 100.0 | 100.0 | 100.0 | 100.0 |
| | Pdl | 0.083 | 0.094 | 0.048 | 0.073 | 0.053 | 0.069 |
| 40° C. 7 d | Size peak 1 | 12.36 | 12.31 | 13.23 | 12.74 | 13.47 | 14.03 |
| | (nm) | | | | | | |
| | Intensity | 100.0 | 100.0 | 92.6 | 100.0 | 100.0 | 100.0 |
| | Pdl | 0.116 | 0.051 | 0.081 | 0.086 | 0.077 | 0.155 |
| 3x FzTh | Size peak 1 | 12.37 | 12.27 | 12.25 | 12.81 | 12.68 | 13.87 |
| | (nm) | | | | | | |
| | Intensity | 100.0 | 100.0 | 100.0 | 100.0 | 100.0 | 100.0 |
| | Pdl | 0.051 | 0.038 | 0.032 | 0.084 | 0.040 | 0.067 |
| Shake | Size peak 1 | 12.56 | 12.37 | 12.35 | 12.61 | 13.29 | 14.63 |
| study | (nm) | | | | | | |
| | Intensity | 100.0 | 100.0 | 100.0 | 100.0 | 100.0 | 100.0 |
| | Pdl | 0.048 | 0.064 | 0.086 | 0.069 | 0.059 | 0.134 |

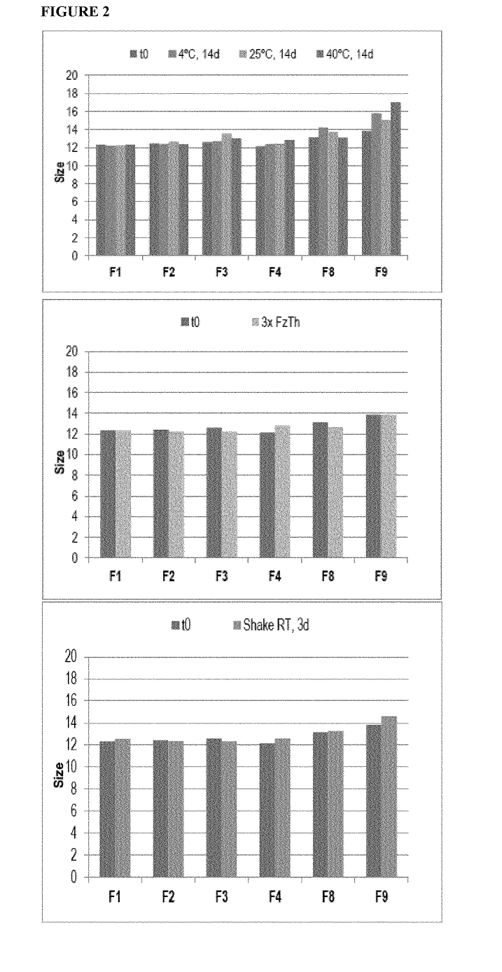

DLS measurements show that the average hydrodynamic particle size does not change significantly from t=0, remaining consistent at all conditions tested for F2, F3, F4 and F8 (see FIG. 2). Even though F9 demonstrates evidence of slightly higher sub visible particulates as compared to the other formulations tested, it maintains a similar purity profile for adalimumab as measured by SDS-PAGE (reduced and non-reduced), SEC-HPLC and IEX-HPLC.

In DLS, 3×FzTh condition, no significant differences in hydrodynamic size noted between formulations and as compared to Innovator. F9 presents a slightly higher particle size overall, but no change from t=0.

### 5. Size Exclusion HPLC Chromatography

The proportion of main peaks, pre-peaks and post-peaks for each of the samples and conditions are shown below:

### HUMIRA Standard:

| | |
|---|---|
| Main peak (%) | 99.73 |
| Pre peak (%) | 0.07 |
| Post peak (%) | 0.19 |

| | F1 t = 0 | F1 25° C. 14 d | F1 40° C. 14 d | F1 shake |
|---|---|---|---|---|
| Main peak (%) | 99.77 | 99.70 | 99.27 | 99.77 |
| Pre peak (%) | 0.05 | 0.04 | 0.06 | 0.04 |
| Post peak (%) | 0.18 | 0.27 | 0.67 | 0.19 |
| | F1 2-8° C. | F1 40° C. 3 d | F1 40° C. 7 d | F1 3x FzTh |
| Main peak (%) | 99.73 | 99.60 | 99.49 | 99.76 |
| Pre peak (%) | | | | |
| Post peak (%) | 0.27 | 0.47 | 0.51 | 0.24 |
| | F2 t = 0 | F2 2-8° C. 14 d | F2 25° C. 14 d | F2 3 d shake |
| Main peak (%) | 99.73 | 99.74 | 99.65 | 99.78 |
| Pre peak (%) | | | | |
| Post peak (%) | 0.27 | 0.26 | 0.35 | 0.22 |
| | F2 40° C. 3 d | F2 40° C. 7 d | F2 40° C. 14 d | F1 3x FzTh |
| Main peak (%) | 99.64 | 99.58 | 99.36 | 99.81 |
| Pre peak (%) | | | | |
| Post peak (%) | 0.36 | 0.42 | 0.64 | 0.19 |
| | F3 t = 0 | F3 2-8° C. 14 d | F3 25° C. 14 d | F3 3 d agitation |
| Main peak (%) | 99.80 | 99.80 | 99.71 | 99.68 |
| Pre peak (%) | | | | |
| Post peak (%) | 0.20 | 0.20 | 0.29 | 0.32 |
| | F3 40° C. 3 d | F3 40° C. 7 d | F3 40° C. 14 d | F3 3x FzTh |
| Main peak (%) | 99.68 | 99.45 | 99.25 | 99.74 |
| Pre peak (%) | | | | |
| Post peak (%) | 0.32 | 0.55 | 0.75 | 0.26 |
| | F4 t = 0 | F4 25° C. 14 d | F4 40° C. 14 d | F4 shake |
| Main peak (%) | 99.79 | 99.68 | 99.38 | 99.80 |
| Pre peak (%) | 0.05 | 0.05 | 0.08 | 0.02 |
| Post peak (%) | 0.16 | 0.27 | 0.55 | 0.17 |
| | F4 2-8° C. | F4 40° C. 3 d | F4 40° C. 7 d | F4 3x FzTh |
| Main peak (%) | 99.74 | 99.66 | 99.56 | 99.77 |
| Pre peak (%) | | | | |
| Post peak (%) | 0.26 | 0.34 | 0.44 | 0.23 |
| | F8 t = 0 | F8 25° C. 14 d | F8 40° C. 14 d | F8 shake |
| Main peak (%) | 99.83 | 99.73 | 99.35 | 99.80 |
| Pre peak (%) | 0.03 | 0.04 | 0.06 | 0.03 |
| Post peak (%) | 0.13 | 0.23 | 0.59 | 0.17 |
| | F8 2-8° C. | F8 40° C. 3 d | F8 40° C. 7 d | F8 3x FzTh |
| Main peak (%) | 99.78 | 99.63 | 99.48 | 99.74 |
| Pre peak (%) | | | | |
| Post peak (%) | 0.22 | 0.37 | 0.52 | 0.26 |
| | F9 t = 0 | F9 25° C. 14 d | F9 40° C. 14 d | F9 shake |
| Main peak (%) | 99.80 | 99.69 | 99.24 | 99.79 |
| Pre peak (%) | 0.04 | 0.03 | 0.02 | 0.02 |
| Post peak (%) | 0.16 | 0.28 | 0.74 | 0.19 |
| | F9 2-8° C. | F9 40° C. 3 d | F9 40° C. 7 d | F9 3x FzTh |
| Main peak (%) | 99.75 | 99.65 | 99.53 | 99.79 |
| Pre peak (%) | | | | |
| Post peak (%) | 0.25 | 0.35 | 0.47 | 0.21 |

Data from SEC-HPLC, at t=0, demonstrated no significant difference between formulations F2, F3, F4, F8 and F9 and as compared to Innovator formulation F1 and HUMIRA reference standard. Aggregate and fragment levels were low for all formulations, with some changes (increase in post-peak fragment) noted for all formulations after 14 days at 40 C, and similar to the Innovator formulation. And this is also shown to be the case for F1 Innovator formulation.

### 6. SDS-PAGE Reduced and Non-Reduced PAGE

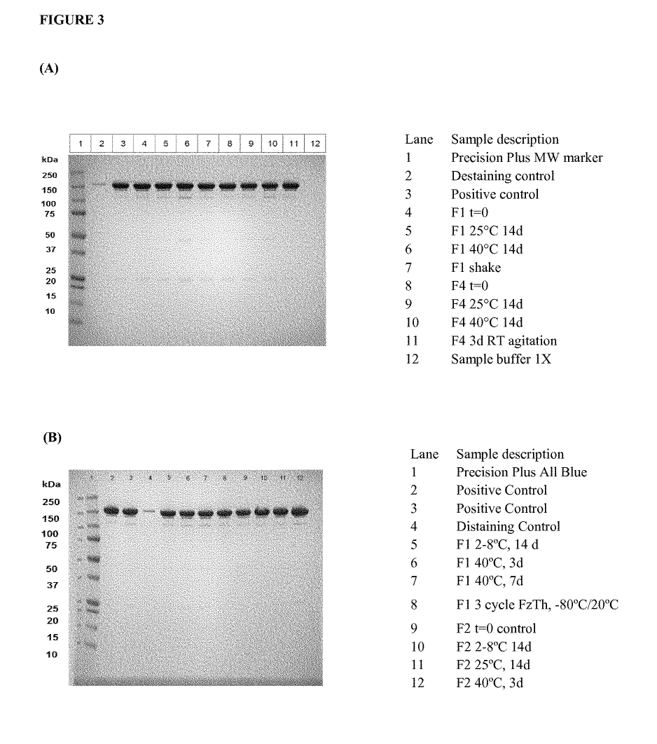

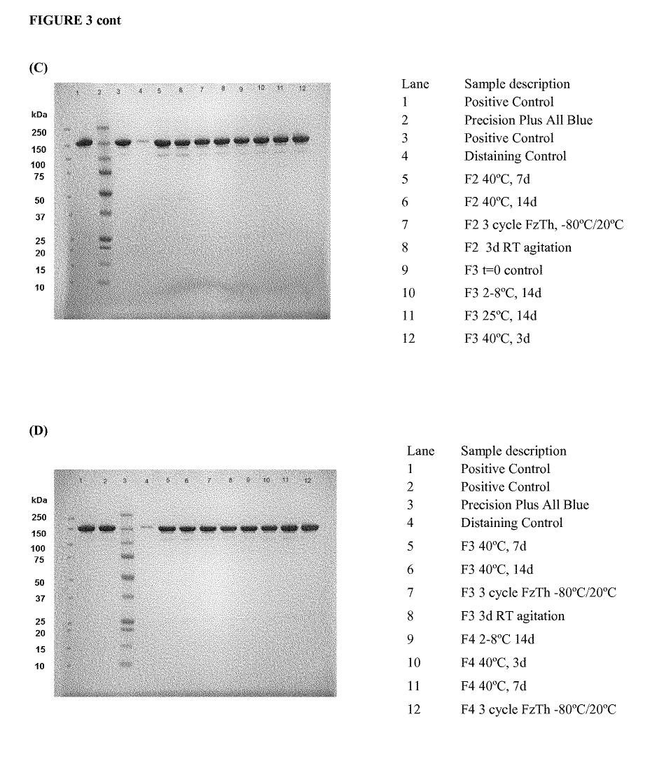

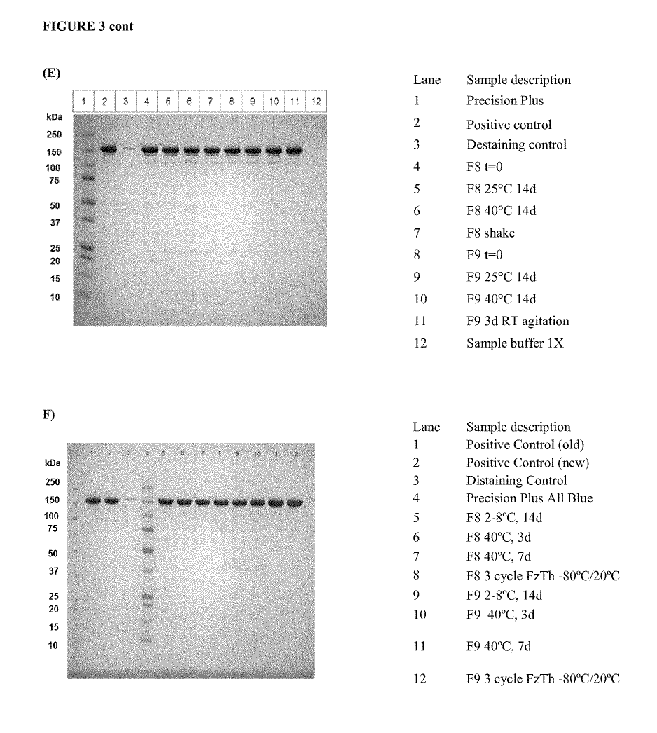

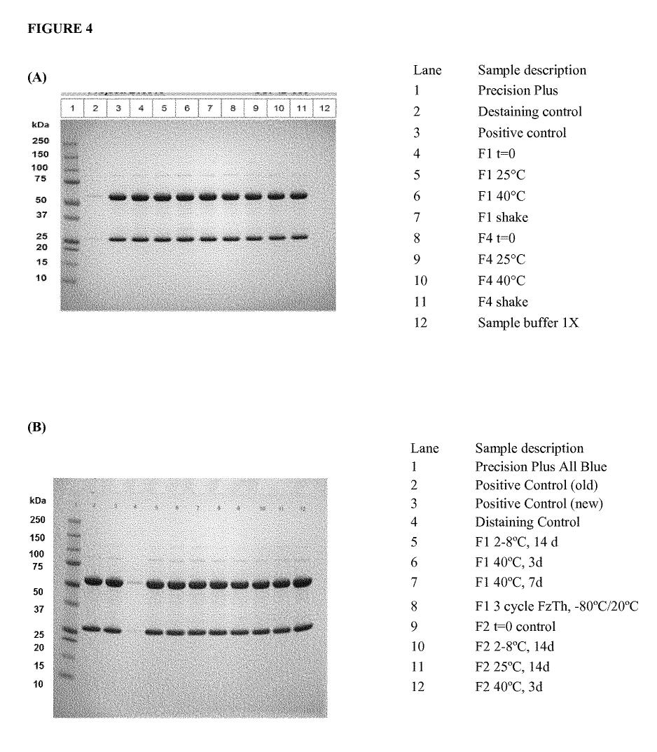

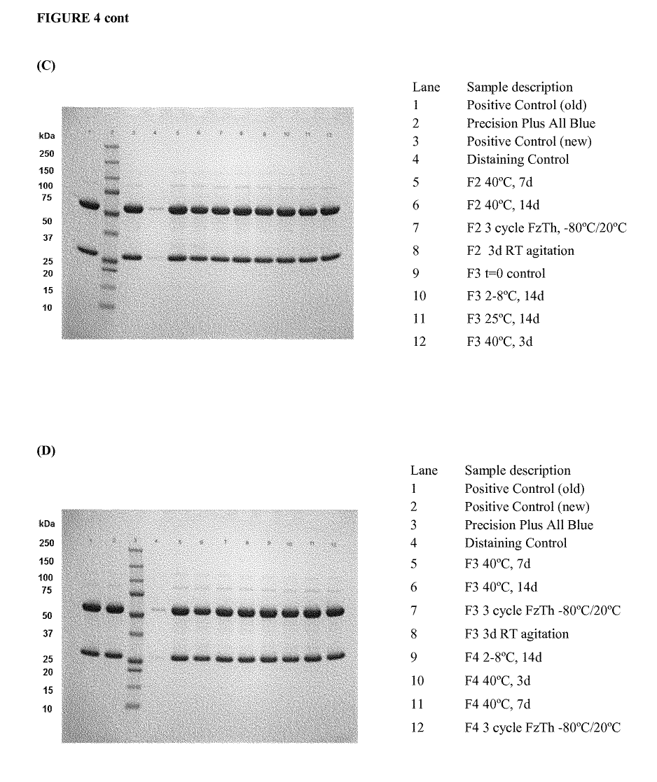

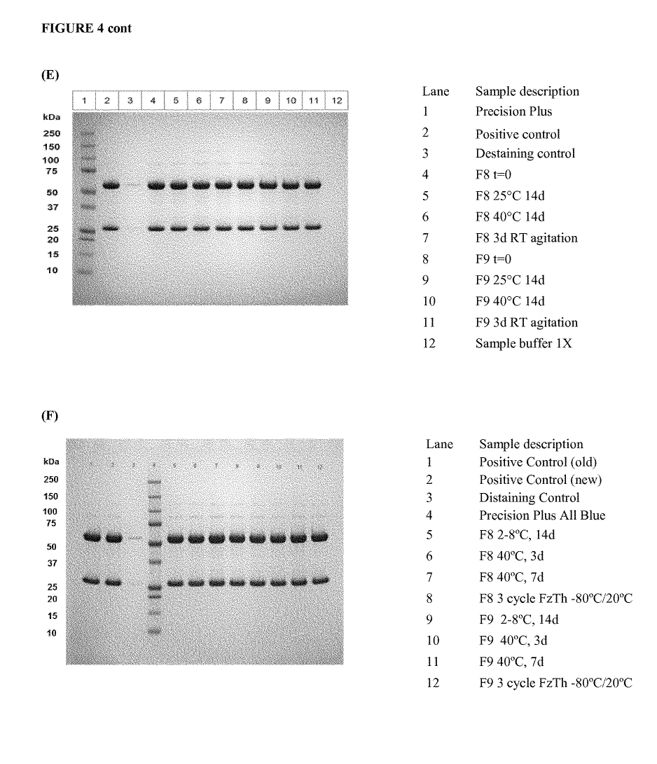

SDS-PAGE under non-reduced conditions, demonstrated no differences between formulations and compared to Innovator formulation at t=0. At the 40° C. condition, increased intensity of the 50 kDa LMW band was evident in all formulations, consistent with the increase in post-peak observed for all formulations by SEC, as seen in FIG. 3.

SDS-PAGE under reduced conditions, demonstrated no differences between formulations and compared to Innovator formulation at t=0 and HUMIRA reference standard (FIG. 3).

SDS-PAGE under reduced conditions at the 40° C. condition, additional LMW breakdown bands were evident for all formulations (FIG. 3).

### 7. IEX-HPLC

The proportion of main peaks, pre-peaks and post-peaks for each of the samples and conditions are shown below:

### HUMIRA Standard:

| | |
|---|---|
| Main peak (%) | 68.6 |
| Pre peak (%) | 7.5 |
| Post peak (%) | 23.9 |

| | F1 t = 0 | F1 2-8° C. 14 d | F1 25° C. 14 d | F1 3 d shake |
|---|---|---|---|---|
| Main peak (%) | 70.9 | 66.3 | 70 | 70.9 |
| Pre peak (%) | 6.4 | 9.3 | 7.6 | 6.5 |
| Post peak (%) | 22.6 | 24.4 | 22.4 | 22.6 |
| | F1 40° C. 3 d | F1 40° C. 7 d | F1 40° C. 14 d | F1 3x FzTh |
| Main peak (%) | 65.1 | 59.9 | 64.3 | |
| Pre peak (%) | 10.2 | 16.7 | 14.5 | |
| Post peak (%) | 24.6 | 23.3 | 21.2 | |
| | F2 t = 0 | F2 2-8° C. 14 d | F2 25° C. 14 d | F2 3 d shake |
| Main peak (%) | 66.4 | 68.19 | 65.9 | 64.112 |
| Pre peak (%) | 9.3 | 9.34 | 9.8 | 13.080 |
| Post peak (%) | 24.3 | 22.46 | 21.3 | 22.808 |
| | F2 40° C. 3 d | F2 40° C. 7 d | F2 40° C. 14 d | F2 3x FzTh |
| Main peak (%) | 66.0 | 60.9 | 56.8 | 65.6 |
| Pre peak (%) | 9.8 | 17.0 | 22.4 | 11.5 |
| Post peak (%) | 24.1 | 22.0 | 99.3 | 22.8 |
| | F3 t = 0 | F3 2-8° C. | F1 25° C. | F3 3 d shake |
| Main peak (%) | 64.1 | 64.2 | 63.1 | 67.5 |
| Pre peak (%) | 13.1 | 13.2 | 14.3 | 10.8 |
| Post peak (%) | 22.7 | 22.5 | 22.4 | 21.6 |
| | F3 40° C. 3 d | F3 40° C. 7 d | F3 40° C. 14 d | F3 3x FzTh |
| Main peak (%) | 62.6 | 63.4 | 62.1 | 67.9 |
| Pre peak (%) | 14.8 | 14.0 | 18.3 | 10.7 |
| Post peak (%) | 22.5 | 22.4 | 19.4 | 21.2 |
| | F4 t = 0 | F4 2-8° C. | F4 25° C. | F4 3 d shake |
| Main peak (%) | 70.2 | 67.8 | 69.6 | 70.1 |
| Pre peak (%) | 7.2 | 10.4 | 8.1 | 7.4 |
| Post peak (%) | 22.6 | 21.6 | 22.4 | 22.5 |
| | F4 40° C. 3 d | F4 40° C. 7 d | F4 40° C. 14 d | F4 3x FzTh |
| Main peak (%) | 67.3 | 63.6 | 60.1 | 67.8 |
| Pre peak (%) | 11.5 | 15.2 | 20.7 | 9.8 |
| Post peak (%) | 21.2 | 21.2 | 19.3 | 22.3 |
| | F8 t = 0 | F8 2-8° C. | F8 25° C. | F8 3 s shake |
| Main peak (%) | 70.7 | 68.2 | 69.6 | 70.6 |
| Pre peak (%) | 6.8 | 9.3 | 8 | 6.6 |
| Post peak (%) | 22.5 | 22.5 | 22.4 | 22.8 |
| | F8 40° C. 3 d | F8 40° C. 7 d | F8 40° C. 14 d | F8 3x FzTh |
| Main peak (%) | 65.2 | 63.2 | 63.3 | 65.941 |
| Pre peak (%) | 11.8 | 14.8 | 13.5 | 10.418 |
| Post peak (%) | 22.8 | 21.9 | 23.2 | 23.641 |
| | F9 t = 0 | F9 2-8° C. | F9 40° C. | F9 3 d agitation |
| Main peak (%) | 70.4 | 67.1 | 70.4 | 68.6 |
| Pre peak (%) | 6.4 | 9.2 | 6.8 | 11 |
| Post peak (%) | 23.2 | 23.7 | 22.8 | 23.9 |
| | F9 40° C. 3 d | F9 40° C. 7 d | F9 40° C. 14 d | F9 3x FzTh |
| Main peak (%) | 65.6 | 63.5 | 65.1 | 66.2 |
| Pre peak (%) | 11.0 | 12.6 | 11 | 9.4 |
| Post peak (%) | 23.3 | 23.9 | 23.9 | 24.5 |

Overall charge variant profile at t=0 as demonstrated by IEX-HPLC was similar for all formulations and as compared to HUMIRA reference standard, and all formulations after 14 days at elevated temperature storage condition showed an increase in % pre-peak (acidic species).

### Example 2

Protein-A purified adalimumab material formulated in the same buffer as HumiraR (hereafter referred to as innovator product) consisting of 6-14 mM sodium citrate/phosphate, 65 mM mannitol, 105 mM NaCl, 0.1% PS80, pH 5.2 (i.e., F1 formulation) was used as starting material. Buffer exchange was performed by dialysis.

The following adalimumab samples in the following buffers were also analyzed, with the concentration of adalimumab being 50 mg/mL in all cases:

Formulation components

F1. Same buffer as Innovator Formulation (sodium chloride, monobasic
sodium phosphate dihydrate, dibasic sodium phosphate dihydrate, sodium
citrate, citric acid monohydrate) pH 5.2, 65 mM mannitol, 0.10% PS80
F2. 20 mM Na Citrate (Na citrate dihydrate, citric acid, monohydrate),
pH 5.2. 65 mM mannitol, 105 mM NaCl, 0.10% PS80
F3. 20 mM Na phosphate (Na phosphate monobasic, monohydrate, Na
phosphate dibasic, anhydrous), pH 6.0, 65 mM mannitol, 105 mM NaCl,
0.10% PS80
F4. 20 mM Histidine acetate (L-Histidine and acetic acid), pH 5.2,
65 mM trehalose, 105 mM NaCl, 0.10% PS80
F8. 20 mM Na acetate (Na acetate anhydrous and acetic acid), pH 5.2,
100 mM sucrose, 105 mM NaCl, 0.10% PS80
F9. 20 mM Na acetate (Na acetate anhydrous and acetic acid), pH 5.2,
100 mM sucrose, 105 mM NaCl, 25 mM Arginine (L-Arginine HCl),
0.10% PS80
F13. 20 mM Na Citrate (Na citrate dihydrate, citric acid,
monohydrate), pH 5.2., 275 mM mannitol, 0.10% PS80
F14. 10 mM Na Citrate (Na citrate dihydrate, citric acid,
monohydrate), pH 5.2., 85 mM mannitol, 105 mM NaCl, 0.10% PS80
F15. 20 mM Na Citrate (Na citrate dihydrate, citric acid,
monohydrate), pH 4.42., 65 mM mannitol, 105 mM NaCl, 0.10% PS80

### Results

1. Visual Appearance at t=0

| | Batch I | | | Batch II | | |
|---|---|---|---|---|---|---|
| | F1 | F2 | F3 | F4 | F8 | F9 |
| Visual | clear, no color, | clear, no color, | clear, no color, | clear, no color, | clear, no color, | clear, no color, |
| Appearance | no particulates | no particulates | no particulates | no particulates | no particulates | no particulates |

Batch III

| | F13 | F14 | F15 |
|---|---|---|---|
| Visual | clear, no color, | clear, no color, | clear, no color, |
| Appearance | no particulates | no particulates | no particulates |

All formulations presented as clear, no color and no visible particulates upon visual inspection and as compared to Innovator formulation.

2. Protein Concentration by A280 nm at t=0

| | Batch I | | | Batch II | | |
|---|---|---|---|---|---|---|
| | F1 | F2 | F3 | F4 | F8 | F9 |
| A280 nm | 0.723 | 0.716 | 0.713 | 0.747 | 0.747 | 0.757 |
| Extrapol. Conc. | 0.516 | 0.511 | 0.509 | 0.534 | 0.534 | 0.541 |
| (mg/mL) | | | | | | |
| Original Conc. | 51.64 | 51.14 | 50.93 | 53.36 | 53.36 | 54.07 |
| (mg/mL) | | | | | | |

| | Batch III | | |
|---|---|---|---|
| | F13 | F14 | F15 |
| A280 nm | 0.721 | 0.675 | 0.749 |
| Extrapol. Conc. | 0.515 | 0.482 | 0.535 |
| (mg/mL) | | | |
| Original Conc. | 51.50 | 48.21 | 53.50 |
| (mg/mL) | | | |

Intrinsic stability of Adalimumab was maintained when exchanged into the alternative buffer and excipient systems selected. Protein concentration remains consistent for all formulations at t=0.

### 3. Sub-Visible Particulate Analysis by DLS

| | Formulation | | | | | | | | | | | |
|---|---|---|---|---|---|---|---|---|---|---|---|---|
| | F1 | | F2 | | F3 | | F4 | | F8 | | F9 | |
| Size (nm) | 12.58 | 142.7 | 12.66 | 144.2 | 12.44 | 125.4 | 12.74 | 151.5 | 13.02 | 163.4 | 13.21 | 175.8 |
| | Peak 1 | Peak 2 | Peak 1 | Peak 2 | Peak 1 | Peak 2 | Peak 1 | Peak 2 | Peak 1 | Peak 2 | Peak 1 | Peak 2 |
| Intensity | 76.9 | 23.1 | 75.9 | 24.1 | 76.8 | 23.2 | 74.9 | 25.1 | 69.8 | 30.2 | 71.0 | 29.0 |
| Pdl | 0.319 | | 0.347 | | 0.373 | | 0.332 | | 0.394 | | 0.375 | |

| Formulation | | |
|---|---|---|
| F13 | F14 | F15 |

| | | | | | | |
|---|---|---|---|---|---|---|
| Size (nm) | 13.08 | 154.2 | 12.74 | 163.8 | 12.42 | 151.7 |
| | Peak 1 | Peak 2 | Peak 1 | Peak 2 | Peak 1 | Peak 2 |
| Intensity | 72.2 | 27.8 | 72.5 | 27.5 | 70.1 | 29.9 |
| Pdl | 0.366 | | 0.370 | | 0.392 | |

DLS measurements show that the average hydrodynamic particle size does not change significantly between formulations for main peak (Peak 1). All formulations, including the Innovator formulation, demonstrate evidence of larger particles (Peak 2 in each case) but with no significant difference between formulations.

### Example 3

### Sample Preparation

Two different drug substance (DS) qualities of the adalimumab biosimilar were used as starting material for this study:
1. A fully purified material, formulated in the same buffer as Humira® (hereafter referred to as innovator product) (6-14 mM sodium citrate/phosphate, 65 mM mannitol, 105 mM NaCl, 0.1% PS80, pH 5.2).
2. A fully purified material, formulated in the same buffer as Humira® but without PS80 (hereafter referred to as innovator product) (6-14 mM sodium citrate/phosphate, 65 mM mannitol, 105 mM NaCl, pH 5.2).

The DS with polysorbate 80 (PS80) was used as starting material for the preparation of F1, F2, F4 and F20 formulations. The PS80 free DS was employed for the preparation of F26, 27 and 28. All formulations were prepared via dialysis.

### Formulations

The following formulations were included for stability and stress testing. All formulations included 50 mg/ml of adalimumab antibody.

| | Buffer | disaccharide | Polyol | Salt | Surfactant |
|---|---|---|---|---|---|
| F2 | 20 mM Na Citrate (Na | 0 | 65 mM | 105 mM | 0.10% |
| | citrate dihydrate, | | mannitol | NaCl | PS80 |
| | citric acid, | | | | |
| | monohydrate) pH 5.2 | | | | |
| F4 | 20 mM His acetate (L- | 65 mM | 0 | 105 mM | 0.10% |
| | Histidine and acetic | trehalose | | NaCl | PS80 |
| | acid) pH 5.2 (MW = | | | | |
| | 215.2 g/mol) | | | | |
| F20 | 20 mM Acetate- Citrate | 0 | 65 mM | 105 mM | 0.10% |
| | (sodium citrate, acetic | | mannitol | NaCl | PS80 |
| | acid) pH 5.2 | | | | |
| F26 | 6-14 mM Na citrate + | 0 | 65 mM | 105 mM | 0.10% |
| | phosphate (sodium | | mannitol | NaCl | PS80 |
| | chloride, monobasic | | | | |
| | sodium phosphate | | | | |
| | dihydrate, dibasic sodium | | | | |
| | phosphate dihydrate, | | | | |
| | sodium citrate, citric | | | | |
| | acid monohydrate) pH 5.2 | | | | |
| F27 | 20 mM Na Citrate (Na | 0 | 65 mM | 105 mM | |
| | citrate dihydrate, citric | | mannitol | NaCl | |
| | acid, monohydrate) pH 5.2 | | | | |
| F28 | 20 mM His acetate (L- | 65 mM | 0 | 105 mM | |
| | Histidine and acetic acid) | trehalose | | NaCl | |
| | pH 5.2 | | | | |

### Tested Conditions

The following conditions were tested on the above samples:

| | | Time |
|---|---|---|
| Condition | Description | point ID |
| Unstressed | Non-stressed formulation after | T0 |
| | preparation | |
| Freeze-thawing | Repetitive freeze-thawing: 5 cycles | T-FT |
| stress | −80° C.-+25° C. Freezing will be | |
| | performed at −80° C. Thawing will | |
| | performed in water bath at 25° C. | |
| | for ca. 0.5 h. | |
| Mechanical or | Shaking at 400 rpm (horizontal | T-mech |
| shaking stress | shaking) for 48 hours at room | |
| | temperature: visual inspection was | |
| | performed after 24, and 48 hours. | |
| | As soon as turbidity/opalescence or | |
| | several visible particles (scores 2 | |
| | to 10) were observed, the stress | |
| | test was stopped. After 48 hours, | |
| | the applied stress was stopped | |
| | independently from the visual | |
| | inspection results. | |
| Storage at 5° C. | 3 months | T-3 mo_5° C. |
| (2-8° C.) | | |
| Storage at 25° C. | 3 months | T-3 mo_25° C. |
| Storage at 40° C. | 1 and 3 months | T-1 mo_40° C. |
| | | T-3 mo_40° C. |

### Results

### 1.—Visual Appearance

The starting material containing PS80 was scored with a value of 1, was slightly turbid and had a slightly yellowish color. DS material formulated PS80-free did not contain any visible particles, resulting in a score of 0.

All formulations investigated did not show any visible particles or only to a very low extent at TO (score of 0 or 1). Upon shaking stress for 24 h, no significant changes were observed for most formulations. However, after shaking for 48 h, the PS80 free formulations F27 and F28 had a high content of visible particles. The other formulations did not show changes after 48 h shaking stress.

Freeze-thaw stress led to formation of some visible particles in F1 and F26 (score of 1). In F4, F20 and F27 the particles were clearly visible within 5 s after freeze-thaw stress. In F28 many visible particles were observed at T-FT (score of 10).

Upon storage only a minor increase of visible particles was observed in samples of F1, F2, F4, F20 and F26, whereas F27 and F28 showed a clear increase of particles in the visible size range upon storage already at 5° C. and 25° C. (scores of 2) and more pronounced at 40° C., resulting in a score of 10 after 1 month and 3 months storage time.

2.—pH

The formulation buffers used for dialysis had a pH of 5.2. The samples after dialysis (TO), as well as at T-mech and T-FT had a pH of 5.2 or 5.3. After storage for 3 months at 40° C., the pH slightly increased to 5.4 for all formulations tested.

### 3.—Osmolality

Osmolality values were between 274-314 mOsmol/kg for all samples (see Table 1). The highest value was measured for F20 (314 mOsmol/kg), the lowest for F28 (274 mOsmol/kg).

TABLE 1

| | | Osmolality | Osmolality of placebo |
|---|---|---|---|
| Formulation | Time point | [mOsmol/kg] | formulation [mOsmol/kg] |
| F1 | T0 | 300 | 313 |
| F2 | T0 | 310 | 314 |
| F4 | T0 | 290 | 288 |
| F20 | T0 | 314 | 326 |
| F26 | T0 | 298 | 302 |
| F27 | T0 | 302 | 316 |
| F28 | T0 | 274 | 274 |

### 4.—UV Spectroscopy

UV spectroscopy was used to determine the protein concentration (λ=280 nm) as well as the aggregation index (A.I.) and turbidity (λ=350 nm).

It was performed on the undiluted samples, at a mAb concentration of ca. 50 mg/ml. For blank measurements, placebo formulations were used.

For most of the samples, the measured concentration of the different formulations remained within 5% of the target value of 50 mg/ml (i.e., between 47.5-52.5 mg/ml). The aggregation index was low for all samples and ranged between 0.01 and 2.27.

### 5.—Micro-Flow Imaging (MFI)

MFI was used to measure subvisible particles in the μm size range. The samples were diluted to 5 mg/ml with filtered placebo formulation prior to measurement. The background was optimized using placebo formulation.

The advantage of MFI in comparison to light obscuration is the ability to characterize and differentiate particles based on their optical properties, such as shape or transparency, which enables to differentiate between silicone oil droplet-like particles and non-silicone oil particles (e.g. proteinaceous particles). With the “find similar” function of the MVAS software the different particle populations can be separated and analyzed. This differentiation, however, is only possible in the range above about 5 μm because of an insufficient resolution of images in the lower size range.

Both bulk materials were investigated by MFI as a short quality check (PS80-containing vs. PS80-free). The overall particle numbers measured by MFI were very low for both materials (up to a few thousand particles ≧1 μm per ml) and free of silicone oil droplet-like particles, which confirms the suitability to perform dialysis with both DS materials.

Overall low particle numbers were detected in F1, F2, F4, F20 and F26, which did not increase much upon mechanical and freeze-thaw stress or storage at different temperatures. In F27 and F28 clearly higher numbers of particles in all size classes (≧1 μm, ≧2 μm, ≧5 μm and ≧10 μm) were measured after mechanical stress.

TABLE 2
MFI results obtained for F1 at different time points.

| | | Cumulative particle numbers per ml | | | | |
|---|---|---|---|---|---|---|
| Time | Particle | | | | ≧10 | ≧25 |
| point | population | ≧1 μm* | ≧2 μm | ≧5 μm | μm | μm |
| T0 | Overall | 2,406 | 612 | 129 | 52 | 7 |
| | Sil. oil | | | 10 | 10 | 0 |
| | Non-sil. oil | | | 118 | 42 | 7 |
| T-mech | Overall | 6,860 | 2,503 | 887 | 424 | 80 |
| | Sil. oil | | | 108 | 0 | 0 |
| | Non-sil. oil | | | 779 | 424 | 80 |
| T-FT | Overall | 7,125 | 1,577 | 399 | 170 | 35 |
| | Sil. oil | | | 0 | 0 | 0 |
| | Non-sil. oil | | | 399 | 170 | 35 |
| T- | Overall | 851 | 160 | 21 | 0 | 0 |
| 1 mo_40° C. | Sil. oil | | | 0 | 0 | 0 |
| | Non-sil. oil | | | 21 | 0 | 0 |
| T- | Overall | 6,338 | 2,482 | 900 | 358 | 83 |
| 3 mo_5° C. | Sil. oil | | | 0 | 0 | 0 |
| | Non-sil. oil | | | 900 | 358 | 83 |
| T- | Overall | 6,425 | 2,281 | 786 | 299 | 66 |
| 3 mo_25° C. | Sil. oil | | | 0 | 0 | 0 |
| | Non-sil. oil | | | 786 | 299 | 66 |
| T- | Overall | 3,028 | 1,464 | 643 | 278 | 59 |
| 3 mo_40° C. | Sil. oil | | | 0 | 0 | 0 |
| | Non-sil. oil | | | 786 | 299 | 66 |

TABLE 3
MFI results obtained for F2 at different time points.

| | | Cumulative particle numbers per ml | | | | |
|---|---|---|---|---|---|---|
| Time | Particle | | | | ≧10 | ≧25 |
| point | population | ≧1 μm* | ≧2 μm | ≧5 μm | μm | μm |
| T0 | Overall | 5,351 | 1,227 | 219 | 38 | 3 |
| | Sil. oil | | | 0 | 0 | 0 |
| | Non-sil. oil | | | 219 | 38 | 3 |
| T-mech | Overall | 14,947 | 4,398 | 1,085 | 327 | 63 |
| | Sil. oil | | | 212 | 3 | 0 |
| | Non-sil. oil | | | 873 | 323 | 63 |
| T-FT | Overall | 6,968 | 4,654 | 382 | 160 | 38 |
| | Sil. oil | | | 0 | 0 | 0 |
| | Non-sil. oil | | | 382 | 160 | 38 |
| T- | Overall | 2,889 | 914 | 261 | 56 | 17 |
| 1 mo_40° C. | Sil. oil | | | 10 | 0 | 0 |
| | Non-sil. oil | | | 250 | 56 | 17 |
| T- | Overall | 2,917 | 1,064 | 407 | 223 | 52 |
| 3 mo_5° C. | Sil. oil | | | 0 | 0 | 0 |
| | Non-sil. oil | | | 407 | 223 | 52 |
| T- | Overall | 4,471 | 1,436 | 445 | 143 | 49 |
| 3 mo_25° C. | Sil. oil | | | 0 | 0 | 0 |
| | Non-sil. oil | | | 445 | 143 | 49 |
| T- | Overall | 4,047 | 1,485 | 435 | 76 | 3 |
| 3 mo_40° C. | Sil. oil | | | 0 | 0 | 0 |
| | Non-sil. oil | | | 435 | 76 | 3 |

TABLE 4
MFI results obtained for F4 at different time points.

| | | Cumulative particle numbers per ml | | | | |
|---|---|---|---|---|---|---|
| Time | Particle | | | | ≧10 | ≧25 |
| point | population | ≧1 μm* | ≧2 μm | ≧5 μm | μm | μm |
| T0 | Overall | 4,502 | 1,150 | 222 | 520 | 17 |
| | Sil. oil | | | 0 | 0 | 0 |
| | Non-sil. oil | | | 222 | 520 | 17 |
| T-mech | Overall | 8,348 | 2,296 | 674 | 267 | 52 |
| | Sil. oil | | | 38 | 0 | 0 |
| | Non-sil. oil | | | 636 | 267 | 52 |
| T-FT | Overall | 14,121 | 3,585 | 823 | 337 | 42 |
| | Sil. oil | | | 0 | 0 | 0 |
| | Non-sil. oil | | | 823 | 337 | 42 |
| T- | Overall | 3,491 | 949 | 156 | 42 | 14 |
| 1 mo_40° C. | Sil. oil | | | 0 | 0 | 0 |
| | Non-sil. oil | | | 156 | 42 | 14 |
| T- | Overall | 3,174 | 1,126 | 546 | 177 | 21 |
| 3 mo_5° C. | Sil. oil | | | 0 | 0 | 0 |
| | Non-sil. oil | | | 546 | 177 | 21 |
| T- | Overall | 7,969 | 2,873 | 1,240 | 434 | 69 |
| 3 mo_25° C. | Sil. oil | | | 0 | 0 | 0 |
| | Non-sil. oil | | | 1,240 | 434 | 69 |
| T- | Overall | 6,988 | 2,646 | 713 | 268 | 59 |
| 3 mo_40° C. | Sil. oil | | | 0 | 0 | 0 |
| | Non-sil. oil | | | 713 | 268 | 59 |

TABLE 5
MFI results obtained for F20 at different time points.

| | | Cumulative particle numbers per ml | | | | |
|---|---|---|---|---|---|---|
| Time | Particle | | | | ≧10 | ≧25 |
| point | population | ≧1 μm* | ≧2 μm | ≧5 μm | μm | μm |
| T0 | Overall | 1,575 | 358 | 76 | 35 | 14 |
| | Sil. oil | | | 0 | 0 | 0 |
| | Non-sil. oil | | | 47 | 35 | 14 |
| T-mech | Overall | 6,039 | 2,138 | 789 | 400 | 90 |
| | Sil. oil | | | 7 | 0 | 0 |
| | Non-sil. oil | | | 782 | 400 | 90 |
| T-FT | Overall | 12,381 | 2,769 | 552 | 149 | 42 |
| | Sil. oil | | | 122 | 10 | 0 |
| | Non-sil. oil | | | 431 | 139 | 42 |
| T- | Overall | 1,499 | 560 | 226 | 90 | 7 |
| 1 mo_40° C. | Sil. oil | | | 10 | 0 | 0 |
| | Non-sil. oil | | | 216 | 90 | 7 |
| T- | Overall | 2,107 | 727 | 299 | 101 | 21 |
| 3 mo_5° C. | Sil. oil | | | 0 | 0 | 0 |
| | Non-sil. oil | | | 299 | 101 | 21 |
| T- | Overall | 1,810 | 778 | 313 | 139 | 24 |
| 3 mo_25° C. | Sil. oil | | | 49 | 14 | 0 |
| | Non-sil. oil | | | 264 | 125 | 24 |
| T- | Overall | 2,097 | 859 | 282 | 104 | 21 |
| 3 mo_40° C. | Sil. oil | | | 0 | 0 | 0 |
| | Non-sil. oil | | | 282 | 104 | 21 |

TABLE 6
MFI results obtained for F26 at different time points.

| | | Cumulative particle numbers per ml | | | | |
|---|---|---|---|---|---|---|
| Time | Particle | | | | ≧10 | ≧25 |
| point | population | ≧1 μm* | ≧2 μm | ≧5 μm | μm | μm |
| T0 | Overall | 1,092 | 278 | 63 | 31 | 7 |
| | Sil. oil | | | 7 | 0 | 0 |
| | Non-sil. oil | | | 56 | 31 | 7 |
| T-mech | Overall | 3,505 | 1,171 | 406 | 174 | 31 |
| | Sil. oil | | | 69 | 10 | 0 |
| | Non-sil. oil | | | 237 | 1163 | 31 |
| T-FT | Overall | 5,846 | 2,050 | 719 | 333 | 45 |
| | Sil. oil | | | 83 | 3 | 0 |
| | Non-sil. oil | | | 363 | 330 | 45 |
| T- | Overall | 1,165 | 396 | 118 | 63 | 14 |
| 1 mo_40° C. | Sil. oil | | | 10 | 0 | 0 |
| | Non-sil. oil | | | 108 | 63 | 14 |
| T- | Overall | 3,828 | 1,761 | 865 | 406 | 35 |
| 3 mo_5° C. | Sil. oil | | | 0 | 0 | 0 |
| | Non-sil. oil | | | 865 | 406 | 35 |
| T- | Overall | 2,510 | 476 | 170 | 101 | 49 |
| 3 mo_25° C. | Sil. oil | | | 0 | 0 | 0 |
| | Non-sil. oil | | | 170 | 101 | 49 |
| T- | Overall | 1,794 | 754 | 292 | 156 | 35 |
| 3 mo_40° C. | Sil. oil | | | 0 | 0 | 0 |
| | Non-sil. oil | | | 292 | 156 | 35 |

TABLE 7
MFI results obtained for F27 at different time points.

| Time | | Cumulative particle numbers per ml | | | | |
|---|---|---|---|---|---|---|
| point | Particle population | ≧1 μm* | ≧2 μm | ≧5 μm | ≧10 μm | ≧25 μm |
| T0 | Overall | 23,886 | 7,608 | 1,678 | 340 | 10 |
| | Sil. oil | | | 28 | 0 | 0 |
| | Non-sil. oil | | | 1,650 | 340 | 0 |
| T-mech | Overall | 255,458 | 71,393 | 19,439 | 4,916 | 841 |
| | Sil. oil | | | 0 | 0 | 0 |
| | Non-sil. oil | | | 19,439 | 4,916 | 841 |
| T-FT | Overall | 21,142 | 4,436 | 393 | 73 | 17 |
| | Sil. oil | | | 0 | 0 | 0 |
| | Non-sil. oil | | | 393 | 73 | 17 |
| T- | Overall | 9,207 | 4,263 | 1,624 | 480 | 80 |
| 1 mo_40° C. | Sil. oil | | | 35 | 0 | 0 |
| | Non-sil. oil | | | 1,589 | 480 | 80 |
| T- | Overall | 8,928 | 4,120 | 1,683 | 462 | 66 |
| 3 mo_5° C. | Sil. oil | | | 0 | 0 | 0 |
| | Non-sil. oil | | | 1,683 | 462 | 66 |
| T- | Overall | 7,315 | 3,859 | 1,613 | 511 | 52 |
| 3 mo_25° C. | Sil. oil | | | 0 | 0 | 0 |
| | Non-sil. oil | | | 1,613 | 511 | 52 |
| T- | Overall | 9,113 | 4,819 | 2,023 | 713 | 153 |
| 3 mo_40° C. | Sil. oil | | | 0 | 0 | 0 |
| | Non-sil. oil | | | 2,023 | 713 | 153 |

TABLE 8
MFI results obtained for F28 at different time points.

| Time | | Cumulative particle numbers per ml | | | | |
|---|---|---|---|---|---|---|
| point | Particle population | ≧1 μm* | ≧2 μm | ≧5 μm | ≧10 μm | ≧25 μm |
| T0 | Overall | 3,825 | 1,423 | 358 | 62 | 4 |
| | Sil. oil | | | 0 | 0 | 0 |
| | Non-sil. oil | | | 358 | 62 | 4 |
| T-mech | Overall | 156,828 | 41,979 | 12,777 | 5,000 | 1,255 |
| | Sil. oil | | | 24 | 7 | 0 |
| | Non-sil. oil | | | 12,753 | 4,993 | 1,255 |
| T-FT | Overall | 4,961 | 1,154 | 355 | 150 | 21 |
| | Sil. oil | | | 31 | 0 | 0 |
| | Non-sil. oil | | | 323 | 150 | 21 |
| T- | Overall | 2,249 | 939 | 417 | 212 | 49 |
| 1 mo_40° C. | Sil. oil | | | 0 | 0 | 0 |
| | Non-sil. oil | | | 417 | 212 | 49 |
| T- | Overall | 2,267 | 994 | 369 | 153 | 42 |
| 3 mo_5° C. | Sil. oil | | | 0 | 0 | 0 |
| | Non-sil. oil | | | 369 | 153 | 42 |
| T- | Overall | 4,120 | 1,964 | 1,189 | 720 | 146 |
| 3 mo_25° C. | Sil. oil | | | 0 | 0 | 0 |
| | Non-sil. oil | | | 1,189 | 720 | 146 |
| T- | Overall | 2,190 | 1,113 | 671 | 386 | 136 |
| 3 mo_40° C. | Sil. oil | | | 0 | 0 | 0 |
| | Non-sil. oil | | | 671 | 386 | 136 |

### 6.—Resonant Mass Measurement (RMM)

ARCHIMEDES quantifies particles in the size range from about 0.3 μm to a few μm based on their weight and density, using resonant mass measurement as a principle. The technique distinguishes between positively buoyant particles (lower density than the formulation, e.g., silicone oil droplets) and negatively buoyant particles (higher density than the formulation, e.g., protein particles). The main purpose of the technique is to detect trends of the particle numbers within the samples (with respect to the different formulations and stress/storage conditions), as well as to determine the ratio between positively (lower density than the formulation, e.g., silicone oil droplets) and negatively buoyant particles (higher density than the formulation, e.g., protein particles). As the analyzed volume is only 150 nl, the technique shows rather trends in the particle amount than determines absolute particle concentrations.

The samples were measured at a concentration of 2.5 mg/ml, after (i) 10 fold dilution with placebo formulation to 5 mg/ml, followed by (ii) 1:1 dilution in with MQ water; the data shown below are calculated back to 5 mg/ml, in order to compare the results with MFI and DLS results, also performed at 5 mg/ml. Negatively buoyant particles with a concentration below or slightly above the limit of quantification (300,000 particles ≧0.3 μm) were detected in most samples at TO, and after storage for 3 months at 5° C., 25° C. and 40° C. F2, F20 and F26 showed higher total particle concentrations, especially after storage at 40° C. The number of positively buoyant was below the limit of quantification of 50 particles for most particles and the values given in the table below are for information only. Furthermore the limit of detection was at a maximal particle size of 0.51-0.56 μm for all samples tested.

TABLE 9
Results of RMM, showing the particle counts for negatively buoyant particles

| | | Limit of | | | | | |
|---|---|---|---|---|---|---|---|
| | | detection | Cumulative particle numbers per ml | | | | |
| Formulation | Time point | [μm] | ≧0.30 μm | ≧0.50 μm | ≧0.75 μm | ≧1.0 μm | ≧2.0 μm |

Negatively buoyant particles

| | | | | | | | |
|---|---|---|---|---|---|---|---|
| F1 | T0 | 0.28 | 313,814 | 91,680 | 38,865 | 25,133 | 2,252 |
| | T-3 mo_5° C. | 0.26 | 192,569 | 67,193 | 27,550 | 12,945 | 0 |
| | T-3 mo_25° C. | 0.26 | 232,458 | 46,0.72 | 13,846 | 13,846 | 4,444 |
| | T-3 mo_40° C. | 0.26 | 88,443 | 9,164 | 4,566 | 0 | 0 |
| F2 | T0 | 0.30 | 400,095 | 79,333 | 42,144 | 18,107 | 2,710 |
| | T-3 mo_5° C. | 0.26 | 100,572 | 48,104 | 23,590 | 8,889 | 0 |
| | T-3 mo_25° C. | 0.26 | 431,111 | 57,778 | 26,667 | 26,667 | 0 |
| | T-3 mo_40° C. | 0.25 | 828,874 | 48,949 | 8,889 | 4,444 | 0 |
| F4 | T0 | 0.32 | 368,880* | 36,275 | 0 | 0 | 0 |
| | T-3 mo_5° C. | 0.26 | 517,838 | 48,949 | 22,282 | 13,393 | 0 |
| | T-3 mo_25° C. | 0.25 | 415,782 | 76,009 | 35,828 | 17,959 | 0 |
| | T-3 mo_40° C. | 0.26 | 179,527 | 37,904 | 18,918 | 4,796 | 0 |
| F20 | T0 | 0.26 | 28,736 | 5,747 | 5,747 | 5,747 | 0 |
| | T-3 mo_5° C. | 0.25 | 429,285 | 67,758 | 9,132 | 9,132 | 0 |
| | T-3 mo_25° C. | 0.26 | 759,224 | 71,529 | 8,949 | 0 | 0 |
| | T-3 mo_40° C. | 0.27 | 959,705 | 54,299 | 13,605 | 0 | 0 |
| F26 | T0 | 0.26 | 407,317 | 140,7.24 | 22,616 | 0 | 0 |
| | T-3 mo_5° C. | 0.26 | 1,190,637 | 251,745 | 80,961 | 27,027 | 0 |
| | T-3 mo_25° C. | 0.26 | 3,880,000 | 1,053,333 | 220,000 | 40,000 | 0 |
| | T-3 mo_40° C. | 0.27 | 1,861,766 | 805,532 | 325,902 | 127,131 | 0 |
| F27 | T0 | 0.26 | 198,124 | 49,216 | 28,650 | 23,922 | 4,728 |
| | T-3 mo_5° C. | 0.27 | 102,168 | 66,205 | 28,717 | 21,470 | 0 |
| | T-3 mo_25° C. | 0.25 | 77,284 | 19,009 | 14,443 | 0 | 0 |
| | T-3 mo_40° C. | 0.26 | 164,387 | 31,878 | 13,578 | 4,474 | 0 |
| F28 | T0 | 0.26 | 153,187 | 27,614 | 13,605 | 4,535 | 0 |
| | T-3 mo_5° C. | 0.25 | 82,563 | 31,008 | 7,752 | 7,752 | 0 |
| | T-3 mo_25° C. | 0.26 | 4,505 | 4,505 | 0 | 0 | 0- |
| | T-3 mo_40° C. | 0.26 | 106,526 | 32,981 | 11.111 | 0 | 0 |

*particle size below the limit of detection, therefore particle concentration might be higher than reported particle number below the limit of quantification of the method (300,000 #/ml); values given in the table above are for information only.

TABLE 10
Results of RMM, showing the particle counts for positively buoyant particles

| | | Limit of | | | | |
|---|---|---|---|---|---|---|
| | | detection | Cumulative particle numbers per ml | | | |
| Formulation | Time point | [μm] | ≧0.50 μm | ≧0.75 μm | ≧1.0 μm | ≧2.0 μm |

Positively buoyant particles

| | | | | | | |
|---|---|---|---|---|---|---|
| F1 | T0 | 0.51 | 9,149* | 2,252 | 0 | 0 |
| | T-3 mo_5° C. | 0.53 | 24,707* | 10,101 | 0 | 0 |
| | T-3 mo_25° C. | 0.52 | 4,866* | 4,866 | 4,866 | 4,866 |
| | T-3 mo_40° C. | 0.52 | 37,900* | 33,333 | 0 | 0 |
| F2 | T0 | 0.54 | 434,406* | 57,133 | 5,442 | 0 |
| | T-3 mo_5° C. | 0.52 | 5,128* | 0 | 0 | 0 |
| | T-3 mo_25° C. | 0.52 | 44,444* | 13,333 | 8,889 | 0 |
| | T-3 mo_40° C. | 0.52 | 35,645* | 8,889 | 8,889 | 0 |
| F4 | T0 | 0.67 | 578,572* | 219,965 | 0 | 0 |
| | T-3 mo_5° C. | 0.52 | 35,736* | 8,889 | 4,444 | 0 |
| | T-3 mo_25° C. | 0.52 | 8,889* | 8,889 | 0 | 0 |
| | T-3 mo_40° C. | 0.53 | 4,598* | 0 | 0 | 0 |
| F20 | T0 | 0.54 | 11,494* | 0 | 0 | 0 |
| | T-3 mo_5° C. | 0.53 | 17,778* | 4,444 | 4,444 | 0 |
| | T-3 mo_25° C. | 0.52 | 0 | 0 | 0 | 0 |
| | T-3 mo_40° C. | 0.54 | 0 | 0 | 0 | 0 |
| F26 | T0 | 0.54 | 17.868* | 4,444 | 4,444 | 0 |
| | T-3 mo_5° C. | 0.52 | 22,742* | 9,195 | 0 | 0 |
| | T-3 mo_25° C. | 0.52 | 60,000* | 13,333 | 6,667 | 0 |
| | T-3 mo_40° C. | 0.56 | 27,216* | 13,671 | 4,474 | 0 |
| F27 | T0 | 0.54 | 0 | 0 | 0 | 0 |
| | T-3 mo_5° C. | 0.55 | 0 | 0 | 0 | 0 |
| | T-3 mo_25° C. | 0.52 | 0 | 0 | 0 | 0 |
| | T-3 mo_40° C. | 0.53 | 0 | 0 | 0 | 0 |
| F28 | T0 | 0.53 | 0 | 0 | 0 | 0 |
| | T-3 mo_5° C. | 0.53 | 0 | 0 | 0 | 0 |
| | T-3 mo_25° C. | 0.52 | 4,505* | 0 | 0 | 0 |
| | T-3 mo_40° C. | 0.52 | 0 | 0 | 0 | 0 |

*particle size below the limit of detection, therefore particle concentration might be higher than reported particle number below the limit of quantification of the method (300,000 #/ml); values given in the table above are for information only.

### 7.—Dynamic Light Scattering (DLS)

Dynamic light scattering analysis enables the determination of the size distribution of aggregates in the nm-size range.

Monoclonal antibodies are generally characterized by a main peak at approximately 10 nm.

Results suggest that F1, F2, F4, F20 and F26 are practically free of nm-sized aggregates since (i) the diameter of the main species corresponds to what expected for monomeric IgG (ii) the intensity of the main species' peak is 100% (iii) the Z-average diameter is almost identical to the diameter of the main species (iv) the PdI values are relatively low.

Aggregates in the nm-size range were measured in all samples of F27 and F28, already at TO. Upon mechanical and freeze-thaw stress, F28 showed a slightly higher main peak content than F27. However, upon storage for 1 month and 3 months the opposite was observed.

TABLE 11
DLS results obtained for F1, F2, F4 and F20 at different
time points, showing Z-average diameters and PdI values.

| Formulation | Time point | Z-average diameter [nm] | PdI |
|---|---|---|---|
| F1 | T0 | 11.24 ± 0.12 | 0.05 ± 0.02 |
| | T-mech | 11.30 ± 0.08 | 0.06 ± 0.00 |
| | T-FT | 11.21 ± 0.08 | 0.05 ± 0.00 |
| | T-1 mo_40° C. | 11.29 ± 0.04 | 0.05 ± 0.01 |
| | T-3 mo_5° C. | 11.22 ± 0.03 | 0.05 ± 0.00 |
| | T-3 mo_25° C. | 11.23 ± 0.12 | 0.06 ± 0.02 |
| | T-3 mo_40° C. | 11.64 ± 0.04 | 0.10 ± 0.02 |
| F2 | T0 | 11.32 ± 0.06 | 0.05 ± 0.01 |
| | T-mech | 11.38 ± 0.07 | 0.06 ± 0.01 |
| | T-FT | 11.32 ± 0.04 | 0.05 ± 0.01 |
| | T-1 mo_40° C. | 11.40 ± 0.03 | 0.07 ± 0.01 |
| | T-3 mo_5° C. | 11.30 ± 0.08 | 0.07 ± 0.01 |
| | T-3 mo_25° C. | 11.21 ± 0.17 | 0.05 ± 0.01 |
| | T-3 mo_40° C. | 11.86 ± 0.14 | 0.11 ± 0.02 |
| F4 | T0 | 11.27 ± 0.09 | 0.08 ± 0.01 |
| | T-mech | 11.36 ± 0.06 | 0.07 ± 0.00 |
| | T-FT | 11.22 ± 0.06 | 0.07 ± 0.01 |
| | T-1 mo_40° C. | 11.24 ± 0.07 | 0.08 ± 0.01 |
| | T-3 mo_5° C. | 11.17 ± 0.10 | 0.08 ± 0.02 |
| | T-3 mo_25° C. | 11.13 ± 0.02 | 0.08 ± 0.02 |
| | T-3 mo_40° C. | 11.55 ± 0.02 | 0.13 ± 0.00 |
| F20 | T0 | 11.27 ± 0.06 | 0.05 ± 0.02 |
| | T-mech | 11.25 ± 0.03 | 0.07 ± 0.01 |
| | T-FT | 11.26 ± 0.09 | 0.06 ± 0.01 |
| | T-1 mo_40° C. | 11.32 ± 0.07 | 0.05 ± 0.01 |
| | T-3 mo_5° C. | 11.11 ± 0.03 | 0.04 ± 0.01 |
| | T-3 mo_25° C. | 11.13 ± 0.02 | 0.05 ± 0.00 |
| | T-3 mo_40° C. | 11.66 ± 0.01 | 0.11 ± 0.01 |

TABLE 12
DLS results obtained for F26-F28 at different time
points, showing Z-average diameters and PdI values.

| Formulation | Time point | Z-average diameter [nm] | PdI |
|---|---|---|---|
| F26 | T0 | 11.38 ± 0.04 | 0.07 ± 0.03 |
| | T-mech | 11.33 ± 0.03 | 0.04 ± 0.00 |
| | T-FT | 11.24 ± 0.04 | 0.04 ± 0.01 |
| | T-1 mo_40° C. | 11.56 ± 0.03 | 0.06 ± 0.00 |
| | T-3 mo_5° C. | 11.17 ± 0.07 | 0.04 ± 0.01 |
| | T-3 mo_25° C. | 11.21 ± 0.06 | 0.06 ± 0.00 |
| | T-3 mo_40° C. | 11.98 ± 0.03 | 0.12 ± 0.01 |
| F27 | T0 | 14.96 ± 2.91 | 0.36 ± 0.17 |
| | T-mech | 14.27 ± 1.53 | 0.32 ± 0.11 |
| | T-FT | 24.78 ± 16.06 | 0.51 ± 0.34 |
| | T-1 mo_40° C. | 14.08 ± 1.04 | 0.30 ± 0.08 |
| | T-3 mo_5° C. | 11.58 ± 0.09 | 0.08 ± 0.02 |
| | T-3 mo_25° C. | 11.69 ± 0.14 | 0.10 ± 0.02 |
| | T-3 mo_40° C. | 14.93 ± 0.56 | 0.35 ± 0.04 |
| F28 | T0 | 12.18 ± 0.31 | 0.17 ± 0.03 |
| | T-mech | 13.91 ± 0.61 | 0.30 ± 0.04 |
| | T-FT | 13.45 ± 0.88 | 0.28 ± 0.07 |
| | T-1 mo_40° C. | 26.37 ± 16.63 | 0.34 ± 0.12 |
| | T-3 mo_5° C. | 12.44 ± 0.30 | 0.23 ± 0.03 |
| | T-3 mo_25° C. | 18.04 ± 5.64 | 0.45 ± 0.15 |
| | T-3 mo_40° C. | 14.60 ± 0.63 | 0.35 ± 0.05 |

8.—Microcalorimetry (g-DSC)

The unfolding temperatures (Tm values) of the protein were determined by g-DSC measurements. Because the reference cell was filled with the corresponding placebo formulation, any change in heat capacity can be related to the protein molecule.

Multiple endothermic transitions were observed upon heating. This is expected and in line with the mAbs' structure, characterized by distinct domains that unfold individually. Typically, the transition with the largest enthalpy of unfolding is caused by the Fab part. Furthermore, two transitions are usually observed for the Fc part, the first one being unfolding of the CH2 domain followed by unfolding of CH3 at higher temperatures.

F4 and F28 exhibited slightly lower Tm values, pointing to higher molecule susceptibility to thermal unfolding, when formulated in 20 mM histidine acetate, pH 5.2, 65 mM trehalose, 105 mM NaCl (with or without PS80). This was particularly true for the lowest transition temperature, Tml. For all the other formulations comparable Tm values were obtained. See table below:

TABLE 13
Results of μ-DSC measurements obtained
for the different formulations at T0.

| Formulation | Tm1 [° C.] | Tm2 [° C.] | Tm3 [° C.] | Tm4 [° C.] |
|---|---|---|---|---|
| F1 | 68.8 | 71.3 | 75.2 | 82.6 |
| F2 | 68.4 | 71.1 | 74.8 | 82.4 |
| F4 | 63.6 | 70.2 | 73.9 | 80.2 |
| F20 | 68.8 | 71.2 | 75.0 | 82.5 |
| F26 | 68.8 | 71.3 | 75.3 | 82.5 |
| F27 | 69.6 | 71.3 | 75.4 | 82.6 |
| F28 | 63.6 | 70.3 | 74.1 | 80.1 |

### 9.—High Performance Size-Exclusion Chromatography (HP-SEC)

HP-SEC was used to assess the purity, monomer content and content of aggregates and fragments of the samples. F4 appears to prevent some monomer loss, after 1 month of storage at 40° C. and 3 months at 5° C. After 3 months of storage at 25° C. all formulations were rather comparable and had approximately 98% monomer content. At higher temperature (40° C.), the monomer and the fragment content in all the formulations, were <92% and about 6-7% respectively. The results are shown in the table below:

TABLE 14
Results obtained by HP-SEC.

| | | Total peak | Monomer | Aggregates | Fragments |
|---|---|---|---|---|---|
| | | area | peak % | peak % | peak % |
| Formulation | Time point | [mAu*min] | area [%] | area [%] | area [%] |
| Form 1 | T0 | 116.2 | 98.7 | 0.6 | 0.7 |
| | T-mech | 134.2 | 98.9 | 0.6 | 0.5 |
| | T-FT | 144.6 | 98.8 | 0.7 | 0.6 |
| | T-1 mo_40° C. | 124.9 | 94.7 | 1.5 | 3.8 |
| | T-3 mo_5° C. | 107.3 | 99.0 | 0.8 | 0.2 |
| | T-3 mo_25° C. | 106.7 | 97.8 | 1.3 | 0.9 |
| | T-3 mo_40° C. | 106.6 | 91.6 | 2.2 | 6.2 |
| Form 2 | T0 | 120.0 | 98.5 | 0.7 | 0.9 |
| | T-mech | 140.4 | 98.9 | 0.6 | 0.5 |
| | T-FT | 159.3 | 98.9 | 0.6 | 0.5 |
| | T-1 mo_40° C. | 129.3 | 94.8 | 1.4 | 3.8 |
| | T-3 mo_5° C. | 113.9 | 98.7 | 0.8 | 0.5 |
| | T-3 mo_25° C. | 112.6 | 97.8 | 1.3 | 0.9 |
| | T-3 mo_40° C. | 111.1 | 91.5 | 2.1 | 6.4 |
| Form 4 | T0 | 122.6 | 98.6 | 0.5 | 0.9 |
| | T-mech | 148.6 | 98.8 | 0.5 | 0.8 |
| | T-FT | 164.8 | 98.6 | 0.4 | 1.1 |
| | T-1 mo_40° C. | 128.1 | 95.7 | 0.9 | 3.4 |
| | T-3 mo_5° C. | 112.5 | 99.2 | 0.5 | 0.3 |
| | T-3 mo_25° C. | 109.5 | 98.3 | 0.8 | 0.9 |
| | T-3 mo_40° C. | 109.1 | 91.5 | 1.4 | 7.1 |
| Form 20 | T0 | 116.7 | 98.7 | 0.6 | 0.7 |
| | T-mech | 135.2 | 98.9 | 0.6 | 0.5 |
| | T-FT | 148.6 | 98.8 | 0.6 | 0.6 |
| | T-1 mo_40° C. | 114.1 | 94.9 | 1.4 | 3.7 |
| | T-3 mo_5° C. | 109.1 | 98.9 | 0.8 | 0.4 |
| | T-3 mo_25° C. | 107.4 | 98.1 | 1.2 | 0.7 |
| | T-3 mo_40° C. | 110.0 | 89.9 | 2.1 | 8.0 |
| Form 26 | T0 | 121.8 | 98.3 | 1.0 | 0.7 |
| | T-mech | 145.9 | 98.3 | 1.1 | 0.6 |
| | T-FT | 132.3 | 98.4 | 1.1 | 0.5 |
| | T-1 mo_40° C. | 133.0 | 94.1 | 2.1 | 3.8 |
| | T-3 mo_5° C. | 111.3 | 98.5 | 1.2 | 0.4 |
| | T-3 mo_25° C. | 109.5 | 97.5 | 1.8 | 0.7 |
| | T-3 mo_40° C. | 109.4 | 89.6 | 3.2 | 7.2 |
| Form 27 | T0 | 117.0 | 98.3 | 0.9 | 0.8 |
| | T-mech | 123.6 | 98.3 | 1.0 | 0.7 |
| | T-FT | 138.2 | 98.5 | 1.0 | 0.6 |
| | T-1 mo_40° C. | 119.6 | 94.1 | 2.0 | 3.9 |
| | T-3 mo_5° C. | 110.1 | 98.4 | 1.1 | 0.5 |
| | T-3 mo_25° C. | 109.3 | 97.6 | 1.6 | 0.8 |
| | T-3 mo_40° C. | 107.0 | 90.3 | 2.8 | 6.9 |
| Form 28 | T0 | 109.1 | 98.5 | 0.7 | 0.9 |
| | T-mech | 128.2 | 98.4 | 0.7 | 0.9 |
| | T-FT | 135.5 | 98.5 | 0.7 | 0.8 |
| | T-1 mo_40° C. | 112.5 | 94.4 | 1.5 | 4.1 |
| | T-3 mo_5° C. | 97.2 | 98.1 | 1.3 | 0.6 |
| | T-3 mo_25° C. | 107.6 | 97.9 | 1.2 | 1.0 |
| | T-3 mo_40° C. | 110.2 | 91.5 | 2.3 | 6.1 |

### 10. Ion-Exchange Chromatography (IEX)

Ion-exchange chromatography was used to evaluate charge variants within the samples.

F26, F27 and F28, which were prepared from the PS80-free DS, featured a lower content of the main isoform than F1, F2, F4 and F20, prepared from the DS containing PS80.

F4 and F28 showed a slightly lower main isoform loss after 1 month of storage at 40° C. and 3 months of storage at 25° C. and 40° C. Under the same storage conditions, F2 was slightly inferior than F20. The results are shown in the table below:

TABLE 15
Results obtained by HP-IEX.

| | | Total | Main | Acidic | Basic |
|---|---|---|---|---|---|
| | | peak area | isoform | isoform | isoform |
| | | [mAu* | peak % | peak % | peak % |
| Formulation | Time point | min] | area [%] | area [%] | area [%] |
| Form 1 | T0 | 117.9 | 62.3 | 18.8 | 19.0 |
| | T-mech | 124.8 | 61.2 | 18.9 | 19.9 |
| | T-FT | 127.1 | 62.1 | 18.6 | 19.4 |
| | T-1 mo_40° C. | 114.3 | 44.1 | 37.3 | 18.6 |
| | T-3 mo_5° C. | 99.2 | 63.4 | 18.3 | 18.2 |
| | T-3 mo_25° C. | 94.7 | 57.2 | 24.3 | 18.5 |
| | T-3 mo_40° C. | 93.0 | 24.1 | 61.5 | 14.4 |
| Form 2 | T0 | 127.5 | 62.3 | 18.9 | 18.9 |
| | T-mech | 132.8 | 61.6 | 18.9 | 19.5 |
| | T-FT | 139.5 | 61.8 | 18.8 | 19.4 |
| | T-1 mo_40° C. | 120.0 | 41.4 | 41.0 | 17.6 |
| | T-3 mo_5° C. | 102.5 | 62.9 | 18.6 | 18.5 |
| | T-3 mo_25° C. | 100.1 | 55.1 | 26.9 | 18.0 |
| | T-3 mo_40° C. | 96.7 | 19.7 | 68.3 | 12.0 |
| Form 4 | T0 | 122.1 | 62.3 | 19.2 | 18.6 |
| | T-mech | 123.8 | 61.6 | 19.2 | 19.2 |
| | T-FT | 140.1 | 61.8 | 19.0 | 19.2 |
| | T-1 mo_40° C. | 114.6 | 46.9 | 33.6 | 19.5 |
| | T-3 mo_5° C. | 100.1 | 62.6 | 19.4 | 18.1 |
| | T-3 mo_25° C. | 99.4 | 58.0 | 23.7 | 18.3 |
| | T-3 mo_40° C. | 94.4 | 29.0 | 55.0 | 16.0 |
| Form 20 | T0 | 121.3 | 62.0 | 18.8 | 19.2 |
| | T-mech | 122.9 | 62.1 | 18.8 | 19.2 |
| | T-FT | 129.1 | 61.7 | 18.8 | 19.5 |
| | T-1 mo_40° C. | 116.5 | 42.7 | 39.3 | 18.1 |
| | T-3 mo_5° C. | 98.9 | 62.7 | 19.2 | 18.1 |
| | T-3 mo_25° C. | 96.8 | 56.0 | 25.9 | 18.1 |
| | T-3 mo_40° C. | 93.7 | 21.3 | 65.7 | 13.0 |
| Form 26 | T0 | 126.8 | 57.5 | 23.7 | 18.8 |
| | T-mech | 119.6 | 57.2 | 23.7 | 19.1 |
| | T-FT | 132.7 | 57.1 | 23.8 | 19.0 |
| | T-1 mo_40° C. | 118.8 | 40.7 | 40.6 | 18.7 |
| | T-3 mo_5° C. | 99.7 | 57.3 | 24.3 | 18.4 |
| | T-3 mo_25°C | 99.2 | 51.8 | 29.9 | 18.3 |
| | T-3 mo_40° C. | 99.0 | 22.5 | 62.8 | 14.7 |
| Form 27 | T0 | 109.7 | 57.2 | 24.0 | 18.8 |
| | T-mech | 111.6 | 56.6 | 24.1 | 19.3 |
| | T-FT | 119.3 | 57.2 | 24.0 | 18.9 |
| | T-1 mo_40° C. | 107.1 | 39.0 | 43.8 | 17.3 |
| | T-3 mo_5° C. | 101.0 | 58.4 | 23.7 | 17.9 |
| | T-3 mo_25° C. | 100.6 | 51.1 | 31.1 | 17.8 |
| | T-3 mo_40° C. | 100.1 | 18.6 | 68.6 | 12.8 |
| Form 28 | T0 | 118.8 | 57.3 | 24.4 | 18.4 |
| | T-mech | 109.1 | 57.1 | 24.0 | 18.9 |
| | T-FT | 123.5 | 57.2 | 24.3 | 18.6 |
| | T-1 mo_40° C. | 108.1 | 42.7 | 38.0 | 19.3 |
| | T-3 mo_5° C. | 105.3 | 57.7 | 24.5 | 17.8 |
| | T-3 mo_25° C. | 101.2 | 55.8 | 29.2 | 15.1 |
| | T-3 mo_40° C. | 94.9 | 25.5 | 57.6 | 17.0 |

1. An aqueous composition comprising:
an anti-TNFα antibody;
a buffer selected from the list consisting of an acetate buffer, a citrate buffer, and a citrate-acetate buffer; and
an excipient, wherein said excipient is at least selected from a disaccharide, a sugar alcohol and a combination thereof;
wherein when the buffer comprises or consists of an acetate buffer, said composition comprises a disaccharide at a concentration of less than 240 mM;
wherein when the buffer comprises or consists of a citrate or a citrate-acetate buffer, said composition comprises a sugar alcohol at a concentration from 50 mM to 300 mM; and
wherein the pH of the composition is from pH 4.0 to pH 7.0.

2. The composition according to claim 1, with the proviso that said composition does not comprise a citrate-phosphate buffer, preferably does not comprise a phosphate buffer.

3. The composition according to any one of claim 1 or 2, comprising:
an anti-TNFα antibody;
a citrate or citrate-acetate buffer;
a sugar alcohol at a concentration from 50 mM to 300 mM; and
optionally, a surfactant;
wherein the pH of the composition is from pH 4.0 to pH 7.0.

4. The composition according to any one of claims 1 to 3, comprising:
an anti-TNFα antibody;
a citrate or citrate-acetate buffer at a concentration from 10 mM to 50 mM;
a sugar alcohol at a concentration from 50 mM to 300 mM; and
optionally, from 0.01% w/v to 1% w/v of a surfactant;
wherein the pH of the composition is from pH 4.0 to pH 7.0.

5. The composition according to any one of claims 1 to 4, wherein the anti-TNFα antibody is a human anti-hTNFα antibody, preferably the anti-TNFα antibody is adalimumab.

6. The composition according to any one of claims 1 to 5, wherein the sugar alcohol is selected from the group consisting of mannitol, xylitol, erythritol, threitol, ribitol, myoinisitol, galactitol, sorbitol, and glycerol, preferably wherein the sugar alcohol is mannitol.

7. The composition according to any one of claims 1 to 6, wherein the sugar alcohol is present at a concentration above 240 mM, preferably at a concentration from 250 mM to 300 mM, more preferably at a concentration of 275 mM.

8. The composition according to any one of claims 1 to 6, wherein the sugar alcohol is present at a concentration below 240 mM, preferably at a concentration below 200 mM.

9. The composition according to any one of claims 1 to 6, wherein the sugar alcohol is present at a concentration from 40 mM to 195 mM, preferably from 50 mM to 100 mM, even more preferably from 55 mM to 90 mM, such as at a concentration of about 65 mM, of about 75 mM or of about 85 mM.

10. The composition according to any one of claims 1 to 9, wherein said buffer is selected from the group consisting of a sodium citrate buffer, a citrate-acetate buffer, a sodium acetate buffer and histidine acetate buffer, preferably is a sodium citrate or a citrate-acetate buffer.

11. The composition according to any one of claims 1 to 10, wherein the pH of the composition is from pH 4.0 to pH 5.7, preferably from pH 4.4 to pH 5.2.

12. The composition according to any one of claims 1 to 11, wherein said composition is substantially free of arginine as a free amino acid.

13. The composition according to any one of claims 1 to 12, wherein the composition further comprises a salt present at a concentration from 80 to 130 mM, preferably wherein the salt concentration is 105 mM, more preferably wherein the salt is sodium chloride.

14. The composition according to any one of claims 1 to 12, wherein the composition further comprises a salt present at a concentration of more than 100 mM, preferably wherein the salt concentration is 105 mM, more preferably wherein the salt is sodium chloride.

15. The composition according to any one of claims 1 to 12, which comprises 10 mg/ml to 200 mg/ml of adalimumab, from 10 mM to 50 mM of a citrate or a citrate-acetate buffer, from 50 to 300 mM of a sugar alcohol and optionally, from 0.05% w/v to 0.2% w/v of a surfactant.

16. The composition according to claim 15, which comprises 10 mg/ml to 200 mg/ml of adalimumab, 10 mM to 50 mM of sodium citrate buffer, from 50 to 300 mM of mannitol and from 0.05% w/v to 0.2% w/v of polysorbate 80.

17. The composition according to claim 16, which comprises 10 mg/ml to 200 mg/ml of adalimumab, 20 mM sodium citrate, 275 mM mannitol, and 0.1% w/v polysorbate 80, wherein the pH of the composition is about pH 5.2.

18. The composition according to any one of claims 1 to 15, wherein when the composition comprises a citrate or a citrate-acetate buffer, the composition further comprises a surfactant.

19. The composition according to any of claims 3 to 15 and 18, wherein the surfactant concentration is from 0.05% w/v to 0.2% w/v, preferably of about 0.1% w/v.

20. The composition according to any one of claims 3 to 15 and 18-19, wherein said surfactant is polysorbate 80.

21. The composition according to any one of claims 1 to 13, which comprises 10 mg/ml to 200 mg/ml of adalimumab, from 10 mM to 50 mM of a citrate or a citrate-acetate buffer, from 50 to 300 mM of a sugar alcohol, from 90 to 115 mM of a salt and optionally from 0.05% w/v to 0.2% w/v of a surfactant.

22. The composition according to claim 21, which comprises 10 mg/ml to 200 mg/ml of adalimumab, 10 mM to 50 mM of citrate or citrate-acetate buffer, from 50 to 300 mM of mannitol, from 90 to 115 mM of NaCl, and optionally from 0.05% w/v to 0.2% w/v of polysorbate 80.

23. The composition according to any one of claim 21 or 22, which comprises 10 mg/ml to 200 mg/ml of adalimumab, 20 mM sodium citrate, 65 mM mannitol, 105 mM NaCl and 0.1% w/v polysorbate 80, wherein the pH of the composition is about pH 5.2.

24. The composition according to any one of claim 21 or 22, which comprises 10 mg/ml to 200 mg/ml of adalimumab, 20 mM sodium citrate, 65 mM mannitol, and 105 mM NaCl, wherein the pH of the composition is about pH 5.2.

25. The composition according to any one of claim 21 or 22, which comprises 10 mg/ml to 200 mg/ml of adalimumab, 10 mM sodium citrate, 85 mM mannitol, 105 mM NaCl and 0.1% w/v polysorbate 80, wherein the pH of the composition is about pH 5.2.

26. The composition according to any one of claim 21 or 22, which comprises 10 mg/ml to 200 mg/ml of adalimumab, 20 mM sodium citrate, 65 mM mannitol, 105 mM NaCl and 0.1% w/v polysorbate 80, wherein the pH of the composition is about pH 4.4, preferably pH 4.42.

27. The composition according to any one of claim 21 or 22, which comprises 10 mg/ml to 200 mg/ml of adalimumab, 20 mM citrate-acetate, 65 mM mannitol, 105 mM NaCl and 0.1% w/v polysorbate 80, wherein the pH of the composition is about pH 5.2.

28. The composition according to any one of claim 21 or 22, which comprises 10 mg/ml to 200 mg/ml of adalimumab, 20 mM citrate-acetate, 65 mM mannitol, and 105 mM NaCl, wherein the pH of the composition is about pH 5.2.

29. The composition according to any one of claim 1 or 2 wherein when the buffer comprises or consists of an acetate buffer, said composition comprises a disaccharide at a concentration of less than 140 mM.

30. The composition according to claim 29 comprising:
an anti-TNFα antibody;
an acetate buffer at a concentration of from 10 mM to 50 mM; and
a disaccharide at a concentration of less than 140 mM; and
optionally, from 0.01% w/v to 1% w/v of a surfactant;
wherein the pH of the composition is from pH 4.0 to pH 7.0.

31. The composition according to any one of claim 29 or 30, wherein the anti-TNFα antibody is a human anti-hTNFα antibody, preferably the anti-TNFα antibody is adalimumab.

32. The composition according to any one of claims 29 to 31, wherein the composition further comprises a salt present at a concentration from 80 to 130 mM, preferably wherein the salt concentration is 105 mM, more preferably wherein the salt is sodium chloride.

33. The composition according to any one of claims 29 to 32, which comprises 10 mg/ml to 200 mg/ml of adalimumab, from 10 mM to 50 mM of an acetate buffer, from 40 mM to 130 mM of a disaccharide, from 90 to 115 mM of a salt and optionally, from 0.05% w/v to 0.2% w/v of a surfactant.

34. The composition according to any one of claims 29 to 33, wherein the disaccharide is trehalose, sucrose or a combination thereof.

35. The composition according to claim 34, wherein when the disaccharide is trehalose, is present at a concentration from 40 mM to 130 mM.

36. The composition according to claim 34, wherein when the disaccharide is sucrose, is present at a concentration from 80 mM to 120 mM, preferably at a concentration of about 100 mM.

37. The composition according to any one of claims 29 to 36, wherein the pH of the composition is from pH 4.0 to pH 5.7, preferably from pH 4.4 to pH 5.2.

38. The composition according to any one of claims 29 to 37, wherein said composition further comprises a surfactant at a concentration from 0.05% w/v to 0.2% w/v, preferably of about 0.1% w/v.

39. The composition according to any one of claims 30 to 38, wherein said surfactant is polysorbate 80.

40. The composition according to any one of claims 29 to 37, wherein said acetate buffer is sodium acetate.

41. The composition according to claim 40, which comprises 10 mg/ml to 200 mg/ml of adalimumab, from 10 mM to 50 mM sodium acetate, from 80 mM to 120 mM sucrose, from 90 to 115 mM NaCl and optionally from 0.05% w/v to 0.2% w/v polysorbate 80.

42. The composition according to claim 41, which comprises 10 mg/ml to 200 mg/ml of adalimumab, 20 mM sodium acetate, 100 mM sucrose, 105 mM NaCl and 0.1% w/v polysorbate 80, wherein the pH of the composition is about pH 5.2.

43. The composition according to claim 37, which comprises 10 mg/ml to 200 mg/ml of adalimumab, from 10 mM to 50 mM sodium acetate, from 80 mM to 120 mM sucrose, from 15 mM to 30 mM Arginine, from 90 to 115 mM NaCl and optionally, from 0.05% w/v to 0.2% w/v polysorbate 80.

44. The composition according to claim 43, comprising 10 mg/ml to 200 mg/ml of adalimumab, 20 mM sodium acetate, 100 mM sucrose, 25 mM Arginine and 105 mM NaCl, wherein the pH of the composition is about pH 5.2.

45. The composition according to any one of claims 29 to 37, wherein said acetate buffer is histidine acetate.

46. The composition according to claim 45, which comprises 10 mg/ml to 200 mg/ml of adalimumab, from 10 mM to 50 mM histidine acetate, from 55 mM to 75 mM trehalose, from 90 to 115 mM NaCl, and optionally from 0.05% w/v to 0.2% w/v polysorbate 80.

47. The composition according to claim 46, which comprises 10 mg/ml to 200 mg/ml of adalimumab, 20 mM histidine acetate, 65 mM trehalose, 105 mM NaCl and 0.1% w/v polysorbate 80, wherein the pH of the composition is about pH 5.2.

48. The composition according to claim 46, which comprises 10 mg/ml to 200 mg/ml of adalimumab, 20 mM histidine acetate, 65 mM trehalose, and 105 mM NaCl, wherein the pH of the composition is about pH 5.2.

49. The composition according to any one of claims 29 to 42 and 45 to 47, wherein said composition is substantially free of arginine as a free amino acid.

50. The composition according to any one of claims 1 to 49, wherein adalimumab is present at a concentration selected from the group consisting of about 10 mg/ml, about 50 mg/ml, about 100 mg/ml, about 150 mg/ml or about 200 mg/ml.

51. The composition according to any one of claims 1 to 50, wherein said composition is a stable composition, preferably wherein said composition is stable at about 25° C. and/or at about 40° C. for at least 14 days, preferably for at least 1 month, more preferably for at least 3 months.

52. The composition according to any one of claims 1 to 51, wherein said composition is a pharmaceutical composition.

53. The composition according to any one of claims 1 to 52, wherein the composition is suitable for administration to a subject via a mode of administration selected from the group consisting of subcutaneous, intravenous, inhalation, intradermal, transdermal, intraperitoneal, and intramuscular.

54. A composition according to any one of claims 1 to 53 for use as a medicament.

55. A device comprising a composition according to any one of claims 1 to 53.

56. The device of claim 55, wherein the device is selected from the group consisting of a syringe, a pen, an implant, a needle-free injection device, an inhalation device, and a patch.

57. A composition according to any one of claims 1 to 53 or a device according to any one of claim 55 or 56 for use in a method for treating inflammatory and/or immune system mediated diseases associated to an increase of TNFα.

58. The composition or device for use according to claim 57, wherein the disease is selected from the group consisting of rheumatoid arthritis, polyarticular juvenile idiopathic arthritis, active enthesitis-related arthritis, psoriatic arthritis, ankylosing spondylitis, Crohn's disease, ulcerative colitis, plaque psoriasis, hidradenitis suppurativa, granulomatosis, chronic obstructive pulmonary disease, hepatitis C, endometriosis, asthma, cachexia, and atopic dermatitis.

59. A method of manufacturing an aqueous composition according to any of claims 1 to 53 which comprises the steps of:
preparing a buffer selected from the list consisting of an acetate buffer, a citrate buffer, or a citrate-acetate buffer at the required pH,
adding a disaccharide and/or sugar alcohol, and optionally, a surfactant and/or salt,
adding an aqueous solution, preferably water, to the final volume and when necessary adjusting the pH,
incorporating an anti-TNFα antibody to the composition.
